(19) United States
(12) Patent Application Publication
(10) Pub. No.: US 2026/0224554 A1
(43) Pub. Date: Aug. 6, 2026

(54) OBEFAZIMOD FOR TREATMENT OF ULCERATIVE COLITIS

(71) Applicant: ABIVAX, Paris (FR)

(72) Inventors: Paul GINESTE, Melun (FR); Anais VISSIAN, Rouen (FR); Mary MANTOCK, Village Neuf (FR)

(73) Assignee: ABIVAX, Paris (FR)

(21) Appl. No.: 19/149,308

(22) PCT Filed: Jul. 30, 2024
(86) PCT No.: PCT/IB2024/000410

§ 371 Date: Jul. 18, 2025

Related U.S. Application Data
(60) Provisional application No. 63/516,910, filed on Aug. 1, 2023.
(60) Provisional application No. 63/580,823, filed on Sep. 6, 2023.

(51) Int. Cl. A61K 31/47; A61P 1/04

(52) U.S. Cl. CPC A61K 31/47; A61P 1/04

(57) ABSTRACT

An obefazimod or a pharmaceutically acceptable salt thereof for use in the treatment of ulcerative colitis.

### CROSS-REFERENCE TO RELATED APPLICATIONS

This application claims the benefit of priority to United States Provisional Patent Application Nos. 63/516,910, filed Aug. 1, 2023 and 63/580,823, filed Sep. 6, 2023, the entirety of each of which is hereby incorporated by reference.

### TECHNICAL FIELD OF THE INVENTION

The present invention relates to use of Obefazimod (also known as ABX464, or 8-chloro-N-[4-(trifluoromethoxy)phenyl]quinolin-2-amine, or (8-chloro-quinolin-2-yl)-(4-trifluoromethoxy-phenyl)-amine), or a pharmaceutically acceptable salt thereof, for treatment of ulcerative colitis. The present invention relates to Obefazimod (also known as ABX464, or 8-chloro-N-[4-(trifluoromethoxy)phenyl]quinolin-2-amine, or (8-chloro-quinolin-2-yl)-(4-trifluoromethoxy-phenyl)-amine), or a pharmaceutically acceptable salt thereof for use in the treatment of ulcerative colitis.

### BACKGROUND OF THE INVENTION

Ulcerative colitis belongs to the group of immune mediated inflammatory diseases (IMIDs) and is characterized by a dysregulated immune response associated with a chronic inflammation of the rectal and colonic mucosa and sub-mucosa layers. The cause of ulcerative colitis is unknown but genetic, environmental and immunologic factors have been proposed as contributing to the IBD pathogenesis.

### SUMMARY OF THE INVENTION

It has been found that certain dosage regimens of Obefazimod provide advantages in treating a patient with ulcerative colitis, including, for example, a patient with moderately to severely active ulcerative colitis.

Accordingly, in one aspect, the present invention provides a method for treating a patient with ulcerative colitis, the method comprising administering to the patient a therapeutically effective amount of Obefazimod, or a pharmaceutically acceptable salt thereof. Accordingly, in one aspect, the present invention provides a therapeutically effective amount of Obefazimod, or a pharmaceutically acceptable salt thereof for use in the treatment of ulcerative colitis in a patient.

In some embodiments, a patient treated with provided methods or provided uses has moderately to severely active ulcerative colitis. In some embodiments, a patient has an inadequate response, no response, a loss of response, or an intolerance to conventional therapies and/or advanced therapies. In some embodiments, a conventional therapy is a corticosteroid or an immunosuppressant. In some embodiments, an immunosuppressant is selected from azathioprine, 6-mercaptopurine, and methotrexate. In some embodiments, an advanced therapy is a biologic, a S1P receptor modulator, or a JAK inhibitor. In some embodiments, a biologic is selected from TNF inhibitors, anti-integrins, and anti-IL-23.

In some embodiments, a method of the present invention comprises administering to a patient Obefazimod, or a pharmaceutically acceptable salt thereof, at an initial dose for an initial period, followed by a subsequent dose for a subsequent period. The present disclosure relates to a therapeutically effective amount of Obefazimod, or a pharmaceutically acceptable salt thereof, at an initial dose for an initial period, followed by a subsequent dose for a subsequent period, for use in the treatment of moderately to severely active ulcerative colitis in a patient. In some embodiments, an initial dose is 50 mg Obefazimod, or a pharmaceutically acceptable salt thereof, once a day. In some embodiments, an initial period is 8 weeks. In some embodiments, a subsequent dose is 25 mg Obefazimod, or a pharmaceutically acceptable salt thereof, once a day. In some embodiments, a subsequent period is 44 weeks.

Some embodiments of the present invention include:
Embodiment 1. A therapeutically effective amount of Obefazimod, or a pharmaceutically acceptable salt thereof, at an initial dose for an initial period, followed by a subsequent dose for a subsequent period, for use in the treatment of moderately to severely active ulcerative colitis in a patient.
Embodiment 2. The therapeutically effective amount of Obefazimod, or a pharmaceutically acceptable salt thereof for use according to claim 1, wherein the initial dose is 50 mg Obefazimod, administered once a day.
Embodiment 3. The therapeutically effective amount of Obefazimod, or a pharmaceutically acceptable salt thereof for use according to claim 1, wherein the initial dose is 25 mg Obefazimod, administered once a day.
Embodiment 4. The therapeutically effective amount of Obefazimod, or a pharmaceutically acceptable salt thereof for use according to any one of claims 1 to 3, wherein the initial period is at least 8 weeks.
Embodiment 5. The therapeutically effective amount of Obefazimod, or a pharmaceutically acceptable salt thereof for use according to any one of claims 1 to 4, wherein the initial period is 8 weeks.
Embodiment 6. The therapeutically effective amount of Obefazimod, or a pharmaceutically acceptable salt thereof for use according to any one of claims 1 to 5, wherein the subsequent dose is 25 mg Obefazimod, administered once a day.
Embodiment 7. The therapeutically effective amount of Obefazimod, or a pharmaceutically acceptable salt thereof for use according to any one of claims 1 to 5, wherein the subsequent dose is 50 mg Obefazimod, administered once a day.
Embodiment 8. The therapeutically effective amount of Obefazimod, or a pharmaceutically acceptable salt thereof for use according to any one of claims 1 to 7, wherein the subsequent period is at least 44 weeks.
Embodiment 9. The therapeutically effective amount of Obefazimod, or a pharmaceutically acceptable salt thereof for use according to any one of claims 1 to 8, wherein the subsequent period is 44 weeks.
Embodiment 10. The therapeutically effective amount of Obefazimod, or a pharmaceutically acceptable salt thereof for use according to any one of claims 1 to 9, wherein the patient has an inadequate response, no response, a loss of response, or an intolerance to conventional therapies and/or advanced therapies for the treatment of moderately to severely active ulcerative colitis. Embodiment 11. The therapeutically effective amount of Obefazimod, or a pharmaceutically acceptable salt thereof for use according to claim 10, wherein the conventional therapy is a corticosteroid or an immunosuppressant.
Embodiment 12. The therapeutically effective amount of Obefazimod, or a pharmaceutically acceptable salt thereof for use according to claim 11, wherein the immunosuppressant is selected from azathioprine, 6-mercaptopurine, and methotrexate.
Embodiment 13. The therapeutically effective amount of Obefazimod, or a pharmaceutically acceptable salt thereof for use according to claim 10, wherein the advanced therapy is a biologic, a S1P receptor modulator, or a JAK inhibitor.
Embodiment 14. The therapeutically effective amount of Obefazimod, or a pharmaceutically acceptable salt thereof for use according to claim 13, wherein the biologic is selected from TNF inhibitors, anti-integrins, and anti-IL-23.
Embodiment 15. A therapeutically effective amount of Obefazimod, or a pharmaceutically acceptable salt thereof for use in the treatment of moderately to severely active ulcerative colitis in a patient, wherein the patient has an inadequate response, no response, a loss of response, or an intolerance to conventional therapies and/or advanced therapies for the treatment of moderately to severely active ulcerative colitis.
Embodiment 16. The therapeutically effective amount of Obefazimod, or a pharmaceutically acceptable salt thereof for use according to claim 15, wherein 50 mg of Obefazimod is administered to the patient once a day.
Embodiment 17. The therapeutically effective amount of Obefazimod, or a pharmaceutically acceptable salt thereof for use according to claim 15, wherein 25 mg of Obefazimod is administered to the patient once a day.
Embodiment 18. The therapeutically effective amount of Obefazimod, or a pharmaceutically acceptable salt thereof for use according to claim 15, wherein Obefazimod, or a pharmaceutically acceptable salt thereof, is administered at an initial dose for an initial period, followed by a subsequent dose for a subsequent period.
Embodiment 19. The therapeutically effective amount of Obefazimod, or a pharmaceutically acceptable salt thereof for use according to claim 18, wherein the initial dose is 50 mg Obefazimod, administered once a day.
Embodiment 20. The therapeutically effective amount of Obefazimod, or a pharmaceutically acceptable salt thereof for use according to claim 18, wherein the initial dose is 25 mg Obefazimod, administered once a day.
Embodiment 21. The therapeutically effective amount of Obefazimod, or a pharmaceutically acceptable salt thereof for use according to any one of claims 18 to 20, wherein the initial period is at least 8 weeks.
Embodiment 22. The therapeutically effective amount of Obefazimod, or a pharmaceutically acceptable salt thereof for use according to any one of claims 18 to 21, wherein the initial period is 8 weeks.
Embodiment 23. The therapeutically effective amount of Obefazimod, or a pharmaceutically acceptable salt thereof for use according to any one of claims 18 to 22, wherein the subsequent dose is 25 mg Obefazimod, administered once a day.
Embodiment 24. The therapeutically effective amount of Obefazimod, or a pharmaceutically acceptable salt thereof for use according to any one of claims 18 to 22, wherein the subsequent dose is 50 mg Obefazimod, administered once a day.
Embodiment 25. The therapeutically effective amount of Obefazimod, or a pharmaceutically acceptable salt thereof for use according to any one of claims 18 to 24, wherein the subsequent period is at least 44 weeks.
Embodiment 26. The therapeutically effective amount of Obefazimod, or a pharmaceutically acceptable salt thereof for use according to any one of claims 18 to 25, wherein the subsequent period is 44 weeks.
Embodiment 27. The therapeutically effective amount of Obefazimod, or a pharmaceutically acceptable salt thereof for use according to any one of claims 15 to 26, wherein the conventional therapy is a corticosteroid or an immunosuppressant.
Embodiment 28. The therapeutically effective amount of Obefazimod, or a pharmaceutically acceptable salt thereof for use according to claim 27, wherein the immunosuppressant is selected from azathioprine, 6-mercaptopurine, and methotrexate.
Embodiment 29. The therapeutically effective amount of Obefazimod, or a pharmaceutically acceptable salt thereof for use according to any one of claims 15 to 26, wherein the advanced therapy is a biologic, a S1P receptor modulator, or a JAK inhibitor.
Embodiment 30. The therapeutically effective amount of Obefazimod, or a pharmaceutically acceptable salt thereof for use according to claim 29, wherein the biologic is selected from TNF inhibitors, anti-integrins, and anti-IL-23.
Embodiment 31. The therapeutically effective amount of Obefazimod, or a pharmaceutically acceptable salt thereof for use according to any one of claims 1 to 30, wherein the patient is administered Obefazimod.
Embodiment 32. The therapeutically effective amount of Obefazimod, or a pharmaceutically acceptable salt thereof for use according to any one of claims 1 to 31, wherein the use has durable therapeutic efficacy over a period greater than 12 months, greater than 18 months, greater than 24 months, greater than 30 months, greater than 36 months, greater than 42 months, greater than 48 months, greater than 54 months, or greater than 60 months.
Embodiment 33. The therapeutically effective amount of Obefazimod, or a pharmaceutically acceptable salt thereof for use according to any one of claims 1 to 32, wherein a subsequent period for administration of a subsequent dose of Obefazimod, results in or maintains a stable modified Mayo score (MMS) in the patient.
Embodiment 34. The therapeutically effective amount of Obefazimod, or a pharmaceutically acceptable salt thereof for use according to any one of claims 1 to 32, wherein a subsequent period for administration of a subsequent dose of Obefazimod, results in or maintains a lower modified Mayo score (MMS) in the patient.
Embodiment 35. The therapeutically effective amount of Obefazimod, or a pharmaceutically acceptable salt thereof for use according to any one of claims 1 to 34, wherein the method results in or maintains a fecal calprotectin level of 250 mcg/g or less in the patient.
Embodiment 36. The therapeutically effective amount of Obefazimod, or a pharmaceutically acceptable salt thereof for use according to any one of claims 1 to 34, wherein during a subsequent period for administration of a subsequent dose of Obefazimod, a patient's fecal calprotectin level is i) maintained at about the same level as the patient's fecal calprotectin level at the beginning of the subsequent period or ii) decreased within a range bounded at the upper end by the patient's fecal calprotectin level at the beginning of the subsequent period and at the lower end at a level about 50% less than the patient's fecal calprotectin level at the beginning of the subsequent period.

### BRIEF DESCRIPTION OF THE DRAWING

FIG. 1 depicts the design of the Phase III study provided in Examples 1 and 2.

FIG. 2 depicts the design of the Phase III study provided in Example 3.

FIG. 3 depicts the design of the Phase II study provided in Example 4. Diamonds under maintenance bar denote annual endoscopy.

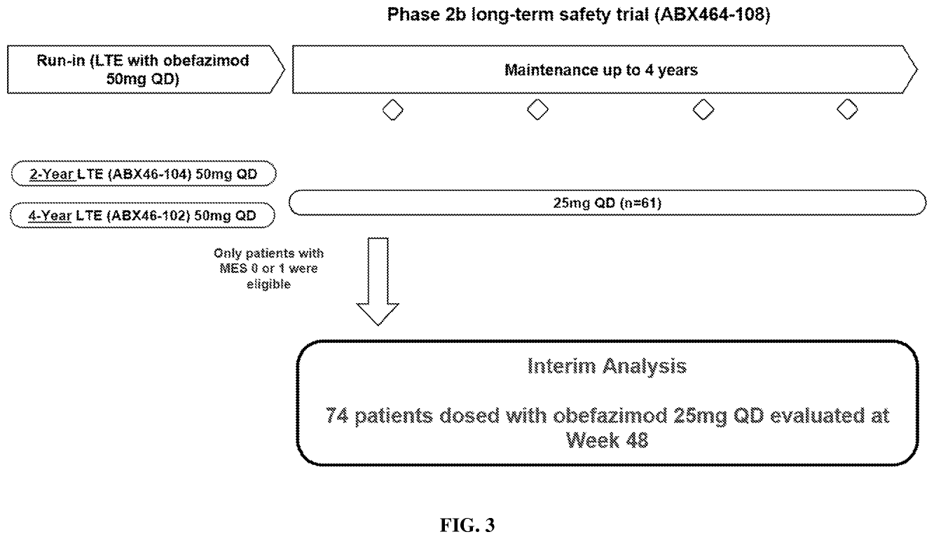

FIG. 4 depicts the disease control rate after 48-week treatment with obefazimod 25 mg QD in the Phase II study of Example 4.

### DETAILED DESCRIPTION OF THE INVENTION

### 1. General Description of Certain Embodiments of the Invention

Obefazimod (also known as ABX464, or 8-chloro-N-[4-(trifluoromethoxy)phenyl]quinolin-2-amine, or (8-chloro-quinolin-2-yl)-(4-trifluoromethoxy-phenyl)-amine) is an oral anti-inflammatory drug candidate. It has been found that Obefazimod specifically upregulates the micro-RNA (miR) 124 and initiates anti-inflammatory downstream effects in preclinical animal models and humans. miR-124 is a critical modulator of immunity and inflammation. miR-124 exerts a crucial role in the development of immune system, regulation of immune responses through directly binding of the 3′UTR of monocyte chemoattractant protein-1 (MCP-1) to dampen inflammation, by affecting macrophage polarization via MCP-1 downregulation and by mediating the cholinergic anti-inflammatory action through inhibiting the production of pro inflammatory cytokines. Overexpression of miR-124 inhibits intestinal inflammation by attenuating the production of interleukin (IL)-6 and tumor necrosis factor (TNF)-α converting enzyme through the targeting of signal transducer and activator of transcription 3 (STAT3) mRNA. The role of miR-124 is essential in inflammatory disorders such as inflammatory bowel diseases (IBD), ulcerative colitis (UC), Crohn's disease (CD) and rheumatoid arthritis (RA).

Accordingly, in one aspect, the present invention provides a method for treating a patient with ulcerative colitis, the method comprising administering to the patient a therapeutically effective amount of Obefazimod, or a pharmaceutically acceptable salt thereof. Accordingly, in one aspect, the present invention provides a therapeutically effective amount of Obefazimod, or a pharmaceutically acceptable salt thereof, for use in the treatment of ulcerative colitis in a patient.

In some embodiments, the present invention provides a method for treating a patient with moderately to severely active ulcerative colitis, the method comprising administering to the patient a therapeutically effective amount of Obefazimod, or a pharmaceutically acceptable salt thereof, wherein the patient has an inadequate response, no response, a loss of response, or an intolerance to conventional therapies and/or advanced therapies. In some embodiments, the present invention provides a therapeutically effective amount of Obefazimod, or a pharmaceutically acceptable salt thereof, for use in the treatment of moderately to severely active ulcerative colitis in a patient, wherein the patient has an inadequate response, no response, a loss of response, or an intolerance to conventional therapies and/or advanced therapies.

In some embodiments, the present invention provides a method for treating a patient with ulcerative colitis, the method comprising administering to the patient Obefazimod, or a pharmaceutically acceptable salt thereof, at an initial dose for an initial period, followed by a subsequent dose for a subsequent period. In some embodiments, the present invention provides a therapeutically effective amount of Obefazimod, or a pharmaceutically acceptable salt thereof, at an initial dose for an initial period, followed by a subsequent dose for a subsequent period, for use in the treatment ulcerative colitis in a patient. In some embodiments, the present invention provides a therapeutically effective amount of Obefazimod, or a pharmaceutically acceptable salt thereof, at an initial dose for an initial period, followed by a subsequent dose for a subsequent period, for use in the treatment of moderately to severely active ulcerative colitis in a patient.

### 2. Definitions

As used herein, the term “Obefazimod” is also known as ABX464, or 8-chloro-N-[4-(trifluoromethoxy)phenyl]quinolin-2-amine, or (8-chloro-quinolin-2-yl)-(4-trifluoromethoxy-phenyl)-amine), having the following formula:

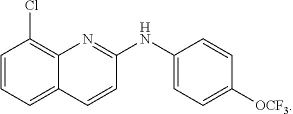 [SMILES: FC(F)(F)Oc1ccc(Nc2ccc3cccc(Cl)c3n2)cc1]

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

As used herein, the term “patient” means an animal, preferably a mammal, and most preferably a human.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, the term “about” shall have the meaning of within 10% of a given value or range. In some embodiments, the term “about” refers to within 10%, 9%, 8%, 7%, 6%, 5%, 4%, 3%, 2%, or 1% of a given value.

### 3. Description of Exemplary Methods and Uses

The present invention encompasses the recognition that Obefazimod may be used to treat a patient with moderately to severely active ulcerative colitis, and which patient has previously had an inadequate response, no response, a loss of response, or an intolerance to conventional therapies and/or advanced therapies. Despite such intolerance or lack of adequate response to other therapies, a patient may be effectively treated for moderately to severely active ulcerative colitis in accordance with the methods provided by the present invention. The present invention also provides particular dosing regimens of Obefazimod that provide advantages (e.g., a reduction in long term or “maintenance” dosing) in treating a patient with ulcerative colitis, including, for example, a patient with moderately to severely active ulcerative colitis. The present invention further provides dosing regimens of Obefazimod that have durable efficacy over extended periods of time.

In some embodiments, the present invention provides a method for treating a patient with moderately to severely active ulcerative colitis, the method comprising administering to the patient a therapeutically effective amount of Obefazimod, or a pharmaceutically acceptable salt thereof, wherein the patient has an inadequate response, no response, a loss of response, or an intolerance to conventional therapies and/or advanced therapies for the treatment of moderately to severely active ulcerative colitis. In some embodiments, the present invention provides a therapeutically effective amount of Obefazimod, or a pharmaceutically acceptable salt thereof, for use in the treatment of moderately to severely active ulcerative colitis in a patient, wherein the patient has an inadequate response, no response, a loss of response, or an intolerance to conventional therapies and/or advanced therapies for the treatment of moderately to severely active ulcerative colitis.

In some embodiments, a therapeutically effective amount of Obefazimod, or a pharmaceutically acceptable salt thereof, is a dose selected from the initial doses and subsequent doses as described herein. In some embodiments, a therapeutically effective amount of Obefazimod, or a pharmaceutically acceptable salt thereof, is 50 mg Obefazimod, or a pharmaceutically acceptable salt thereof (e.g., in an amount equivalent to 50 mg Obefazimod as the active pharmaceutical ingredient), once a day. In some embodiments, a therapeutically effective amount of Obefazimod, or a pharmaceutically acceptable salt thereof, is 25 mg Obefazimod, or a pharmaceutically acceptable salt thereof (e.g., in an amount equivalent to 25 mg Obefazimod as the active pharmaceutical ingredient), once a day.

In some embodiments, the present invention provides a method for treating a patient with moderately to severely active ulcerative colitis, the method comprising administering to the patient a therapeutically effective amount of Obefazimod, or a pharmaceutically acceptable salt thereof, wherein the method has durable therapeutic efficacy over a period of at least 12 months, at least 18 months, at least 24 months, at least 30 months, at least 36 months, at least 42 months, at least 48 months, at least 54 months, or at least 60 months. In some embodiments, the present invention provides a therapeutically effective amount of Obefazimod, or a pharmaceutically acceptable salt thereof for use in the treatment of moderately to severely active ulcerative colitis in a patient, wherein the use has durable therapeutic efficacy over a period of at least 12 months, at least 18 months, at least 24 months, at least 30 months, at least 36 months, at least 42 months, at least 48 months, at least 54 months, or at least 60 months. In some embodiments, the present invention provides a method for treating a patient with moderately to severely active ulcerative colitis, the method comprising administering to the patient a therapeutically effective amount of Obefazimod, or a pharmaceutically acceptable salt thereof, wherein the method has durable therapeutic efficacy over a period greater than 12 months, greater than 18 months, greater than 24 months, greater than 30 months, greater than 36 months, greater than 42 months, greater than 48 months, greater than 54 months, or greater than 60 months. In some embodiments, the present invention provides a therapeutically effective amount of Obefazimod, or a pharmaceutically acceptable salt thereof for use in the treatment of moderately to severely active ulcerative colitis in a patient, wherein the use has durable therapeutic efficacy over a period greater than 12 months, greater than 18 months, greater than 24 months, greater than 30 months, greater than 36 months, greater than 42 months, greater than 48 months, greater than 54 months, or greater than 60 months. In some embodiments, the present invention provides a method for treating a patient with moderately to severely active ulcerative colitis, the method comprising administering to the patient a therapeutically effective amount of Obefazimod, or a pharmaceutically acceptable salt thereof, wherein the method has durable therapeutic efficacy over a period greater than 12 months. In some embodiments, the present invention provides a therapeutically effective amount of Obefazimod, or a pharmaceutically acceptable salt thereof, for use in the treatment of moderately to severely active ulcerative colitis in a patient, wherein the use has durable therapeutic efficacy over a period greater than 12 months. In some embodiments, the present invention provides a method for treating a patient with moderately to severely active ulcerative colitis, the method comprising administering to the patient a therapeutically effective amount of Obefazimod, or a pharmaceutically acceptable salt thereof, wherein the method has durable therapeutic efficacy over a period greater than 18 months. In some embodiments, the present invention provides a therapeutically effective amount of Obefazimod, or a pharmaceutically acceptable salt thereof, for use in the treatment of moderately to severely active ulcerative colitis in a patient, wherein the use has durable therapeutic efficacy over a period greater than 18 months. In some embodiments, the present invention provides a method for treating a patient with moderately to severely active ulcerative colitis, the method comprising administering to the patient a therapeutically effective amount of Obefazimod, or a pharmaceutically acceptable salt thereof, wherein the method has durable therapeutic efficacy over a period greater than 24 months. In some embodiments, the present invention provides a therapeutically effective amount of Obefazimod, or a pharmaceutically acceptable salt thereof, for use in the treatment of moderately to severely active ulcerative colitis in a patient, wherein the use has durable therapeutic efficacy over a period greater than 24 months. In some embodiments, the present invention provides a method for treating a patient with moderately to severely active ulcerative colitis, the method comprising administering to the patient a therapeutically effective amount of Obefazimod, or a pharmaceutically acceptable salt thereof, wherein the method has durable therapeutic efficacy over a period greater than 30 months. In some embodiments, the present invention provides a therapeutically effective amount of Obefazimod, or a pharmaceutically acceptable salt thereof, for use in the treatment of moderately to severely active ulcerative colitis in a patient, wherein the use has durable therapeutic efficacy over a period greater than 30 months. In some embodiments, the present invention provides a method for treating a patient with moderately to severely active ulcerative colitis, the method comprising administering to the patient a therapeutically effective amount of Obefazimod, or a pharmaceutically acceptable salt thereof, wherein the method has durable therapeutic efficacy over a period greater than 36 months. In some embodiments, the present invention provides a therapeutically effective amount of Obefazimod, or a pharmaceutically acceptable salt thereof, for use in the treatment of moderately to severely active ulcerative colitis in a patient, wherein the use has durable therapeutic efficacy over a period greater than 36 months. In some embodiments, the present invention provides a method for treating a patient with moderately to severely active ulcerative colitis, the method comprising administering to the patient a therapeutically effective amount of Obefazimod, or a pharmaceutically acceptable salt thereof, wherein the method has durable therapeutic efficacy over a period greater than 42 months. In some embodiments, the present invention provides a therapeutically effective amount of Obefazimod, or a pharmaceutically acceptable salt thereof, for use in the treatment of moderately to severely active ulcerative colitis in a patient, wherein the use has durable therapeutic efficacy over a period greater than 42 months. In some embodiments, the present invention provides a method for treating a patient with moderately to severely active ulcerative colitis, the method comprising administering to the patient a therapeutically effective amount of Obefazimod, or a pharmaceutically acceptable salt thereof, wherein the method has durable therapeutic efficacy over a period greater than 48 months. In some embodiments, the present invention provides a therapeutically effective amount of Obefazimod, or a pharmaceutically acceptable salt thereof, for use in the treatment of moderately to severely active ulcerative colitis in a patient, wherein the use has durable therapeutic efficacy over a period greater than 48 months. In some embodiments, the present invention provides a method for treating a patient with moderately to severely active ulcerative colitis, the method comprising administering to the patient a therapeutically effective amount of Obefazimod, or a pharmaceutically acceptable salt thereof, wherein the method has durable therapeutic efficacy over a period greater than 54 months. In some embodiments, the present invention provides a therapeutically effective amount of Obefazimod, or a pharmaceutically acceptable salt thereof, for use in the treatment of moderately to severely active ulcerative colitis in a patient, wherein the use has durable therapeutic efficacy over a period greater than 54 months. In some embodiments, the present invention provides a method for treating a patient with moderately to severely active ulcerative colitis, the method comprising administering to the patient a therapeutically effective amount of Obefazimod, or a pharmaceutically acceptable salt thereof, wherein the method has durable therapeutic efficacy over a period greater than 60 months. In some embodiments, the present invention provides a therapeutically effective amount of Obefazimod, or a pharmaceutically acceptable salt thereof, for use in the treatment of moderately to severely active ulcerative colitis in a patient, wherein the use has durable therapeutic efficacy over a period greater than 60 months.

In some embodiments, the present invention provides a method for treating a patient with moderately to severely active ulcerative colitis, the method comprising administering to the patient a therapeutically effective amount of Obefazimod, or a pharmaceutically acceptable salt thereof, at an initial dose for an initial period, followed by a subsequent dose for a subsequent period. In some embodiments, the present invention provides a therapeutically effective amount of Obefazimod, or a pharmaceutically acceptable salt thereof, at an initial dose for an initial period, followed by a subsequent dose for a subsequent period, for use in the treatment of moderately to severely active ulcerative colitis in a patient. In some embodiments, a patient has an inadequate response, no response, a loss of response, or an intolerance to conventional therapies and/or advanced therapies for the treatment of moderately to severely active ulcerative colitis.

In some embodiments, a conventional therapy is a corticosteroid or an immunosuppressant. In some embodiments, an immunosuppressant is selected from azathioprine, 6-mercaptopurine, and methotrexate. In some embodiments, an advanced therapy is a biologic, a S1P receptor modulator, or a JAK inhibitor. In some embodiments, a biologic is selected from TNF inhibitors, anti-integrins, and anti-IL-23.

In some embodiments, Obefazimod, or a pharmaceutically acceptable salt thereof, is administered with food. In some embodiments, Obefazimod, or a pharmaceutically acceptable salt thereof, is administered with food in the morning. In some embodiments, Obefazimod, or a pharmaceutically acceptable salt thereof, is administered with food and water in the morning. In some embodiments, Obefazimod, or a pharmaceutically acceptable salt thereof, is administered in a fed condition. In some embodiments, Obefazimod, or a pharmaceutically acceptable salt thereof, is administered in a fed condition in the morning. In some embodiments, Obefazimod, or a pharmaceutically acceptable salt thereof, is administered in a fed condition in the morning with water.

### Initial Dose

In some embodiments, an initial dose, as provided in a method or in a use of the present invention, is 50 mg Obefazimod, or a pharmaceutically acceptable salt thereof, once a day. In some embodiments, an initial dose, as provided in a method or in a use of the present invention, is 25 mg Obefazimod, or a pharmaceutically acceptable salt thereof, once a day.

### Initial Period

An initial period, during which Obefazimod, or a pharmaceutically acceptable salt thereof, is administered at an initial dose, as provided in a method or in a use of the present invention, can range from one week to three years. In some embodiments, an initial period for administration of an initial dose of Obefazimod, or a pharmaceutically acceptable salt thereof, ranges from one week to six weeks. In some embodiments, an initial period for administration of an initial dose of Obefazimod, or a pharmaceutically acceptable salt thereof, ranges from one week to eight weeks. In some embodiments, an initial period for administration of an initial dose of Obefazimod, or a pharmaceutically acceptable salt thereof, ranges from six weeks to three months. In some embodiments, an initial period for administration of an initial dose of Obefazimod, or a pharmaceutically acceptable salt thereof, ranges from three months to six months. In some embodiments, an initial period for administration of an initial dose of Obefazimod, or a pharmaceutically acceptable salt thereof, ranges from six months to one year. In some embodiments, an initial period for administration of an initial dose of Obefazimod, or a pharmaceutically acceptable salt thereof, ranges from one year to three years. In some embodiments, an initial period for administration of an initial dose of Obefazimod, or a pharmaceutically acceptable salt thereof, ranges from one year to two years. In some embodiments, an initial period for administration of an initial dose of Obefazimod, or a pharmaceutically acceptable salt thereof, ranges from two years to three years. In some embodiments, an initial period for administration of an initial dose of Obefazimod, or a pharmaceutically acceptable salt thereof, ranges from three years to four years.

In some embodiments, an initial period for administration of an initial dose of Obefazimod, or a pharmaceutically acceptable salt thereof, as provided in a method or in a use of the present invention, is at least 8 weeks. In some embodiments, an initial period for administration of an initial dose of Obefazimod, or a pharmaceutically acceptable salt thereof, as provided in a method or in a use of the present invention, is 8 weeks. In some embodiments, an initial period for administration of an initial dose of Obefazimod, or a pharmaceutically acceptable salt thereof, as provided in a method or in a use of the present invention, is one year. In some embodiments, an initial period for administration of an initial dose of Obefazimod, or a pharmaceutically acceptable salt thereof, as provided in a method or in a use of the present invention, is two years. In some embodiments, an initial period for administration of an initial dose of Obefazimod, or a pharmaceutically acceptable salt thereof, as provided in a method or in a use of the present invention, is three years. In some embodiments, an initial period for administration of an initial dose of Obefazimod, or a pharmaceutically acceptable salt thereof, as provided in a method or in a use of the present invention, is four years.

### Subsequent Dose

In some embodiments, a subsequent dose, as provided in a method or in a use of the present invention, is 25 mg Obefazimod, or a pharmaceutically acceptable salt thereof, once a day. In some embodiments, a subsequent dose, as provided in a method or in a use of the present invention, is 50 mg Obefazimod, or a pharmaceutically acceptable salt thereof, once a day.

### Subsequent Period

A subsequent period, during which Obefazimod, or a pharmaceutically acceptable salt thereof, is administered at a subsequent dose, as provided in a method or in a use of the present invention, can range from one week to three years. In some embodiments, a subsequent period for administration of a subsequent dose of Obefazimod, or a pharmaceutically acceptable salt thereof, ranges from one week to six weeks. In some embodiments, a subsequent period for administration of a subsequent dose of Obefazimod, or a pharmaceutically acceptable salt thereof, ranges from six weeks to three months. In some embodiments, a subsequent period for administration of a subsequent dose of Obefazimod, or a pharmaceutically acceptable salt thereof, ranges from three months to six months. In some embodiments, a subsequent period for administration of a subsequent dose of Obefazimod, or a pharmaceutically acceptable salt thereof, ranges from six months to one year. In some embodiments, a subsequent period for administration of a subsequent dose of Obefazimod, or a pharmaceutically acceptable salt thereof, ranges from one year to three years. In some embodiments, a subsequent period for administration of a subsequent dose of Obefazimod, or a pharmaceutically acceptable salt thereof, is three or more years. In some embodiments, a subsequent period for administration of a subsequent dose of Obefazimod, or a pharmaceutically acceptable salt thereof, is until a physician determines that the dose should be adjusted or that administration of Obefazimod should be terminated.

In some embodiments, a subsequent period for administration of a subsequent dose of Obefazimod, or a pharmaceutically acceptable salt thereof, as provided in a method or in a use of the present invention, is at least 44 weeks. In some embodiments, a subsequent period for administration of a subsequent dose of Obefazimod, or a pharmaceutically acceptable salt thereof, as provided in a method or in a use of the present invention, is 44 weeks. In some embodiments, a subsequent period for administration of a subsequent dose of Obefazimod, or a pharmaceutically acceptable salt thereof, as provided in a method or in a use of the present invention, is 48 weeks.

In some embodiments, a subsequent period for administration of a subsequent dose of Obefazimod, or a pharmaceutically acceptable salt thereof, as provided in a method or in a use of the present invention, results in or maintains the same modified Mayo score (MMS) in a patient. In some embodiments, the same modified Mayo score (MMS) in a patient refers to the same MMS throughout the subsequent period. In some embodiments, the same modified Mayo score (MMS) in a patient refers to the same MMS at the end of the subsequent period as compared to the beginning of the subsequent period. In some embodiments, a subsequent period for administration of a subsequent dose of Obefazimod, or a pharmaceutically acceptable salt thereof, as provided in a method or in a use of the present invention, results in or maintains an improved (e.g., lower) modified Mayo score (MMS) in a patient. In some embodiments, an improved (e.g., lower) modified Mayo score (MMS) in a patient refers to an improved (e.g., lower) or improving MMS throughout the subsequent period. In some embodiments, an improved (e.g., lower) modified Mayo score (MMS) in a patient refers to an improved (e.g., lower) MMS at the end of the subsequent period as compared to the beginning of the subsequent period.

In some embodiments, a subsequent period for administration of a subsequent dose of Obefazimod, or a pharmaceutically acceptable salt thereof, as provided in a method or in a use of the present invention, results in or maintains a fecal calprotectin level of 500 microgm/gm (hereafter: mcg/g) or less in a patient. In some embodiments, a subsequent period for administration of a subsequent dose of Obefazimod, or a pharmaceutically acceptable salt thereof, as provided in a method or in a use of the present invention, results in or maintains a fecal calprotectin level of about 475 mcg/g, about 450 mcg/g, about 425 mcg/g, about 400 mcg/g, about 375 mcg/g, about 350 mcg/g, about 325 mcg/g, about 300 mcg/g, or about 275 mcg/g in a patient. In some embodiments, a subsequent period for administration of a subsequent dose of Obefazimod, or a pharmaceutically acceptable salt thereof, as provided in a method or in a use of the present invention, results in or maintains a fecal calprotectin level of 250 mcg/g or less in a patient. In some embodiments, a subsequent period for administration of a subsequent dose of Obefazimod, or a pharmaceutically acceptable salt thereof, as provided in a method or in a use of the present invention, results in or maintains a fecal calprotectin level of about 240 mcg/g, about 230 mcg/g, about 220 mcg/g, about 210 mcg/g, about 200 mcg/g, about 190 mcg/g, about 180 mcg/g, about 170 mcg/g, about 160 mcg/g, about 150 mcg/g, about 140 mcg/g, about 130 mcg/g, about 120 mcg/g, or about 110 mcg/g in a patient. In some embodiments, a subsequent period for administration of a subsequent dose of Obefazimod, or a pharmaceutically acceptable salt thereof, as provided in a method or in a use of the present invention, results in or maintains a fecal calprotectin level of 100 mcg/g or less in a patient.

In some embodiments, during a subsequent period for administration of a subsequent dose of Obefazimod, or a pharmaceutically acceptable salt thereof, as provided in a method or in a use of the present invention, a patient's fecal calprotectin level is maintained at a level within about 20% of the patient's fecal calprotectin level at the beginning of the subsequent period. In some embodiments, during a subsequent period for administration of a subsequent dose of Obefazimod, or a pharmaceutically acceptable salt thereof, as provided in a method or in a use of the present invention, a patient's fecal calprotectin level is maintained at a level within about 10% of the patient's fecal calprotectin level at the beginning of the subsequent period. In some embodiments, during a subsequent period for administration of a subsequent dose of Obefazimod, or a pharmaceutically acceptable salt thereof, as provided in a method or in a use of the present invention, a patient's fecal calprotectin level is maintained at a level within about 5% of the patient's fecal calprotectin level at the beginning of the subsequent period.

In some embodiments, during a subsequent period for administration of a subsequent dose of Obefazimod, or a pharmaceutically acceptable salt thereof, as provided in a method or in a use of the present invention, a patient's fecal calprotectin level is i) maintained at about the same level as the patient's fecal calprotectin level at the beginning of the subsequent period or ii) decreased within a range bounded at the upper end by the patient's fecal calprotectin level at the beginning of the subsequent period and at the lower end at a level about 50% less than the patient's fecal calprotectin level at the beginning of the subsequent period. In some embodiments, during a subsequent period for administration of a subsequent dose of Obefazimod, or a pharmaceutically acceptable salt thereof, as provided in a method or in a use of the present invention, a patient's fecal calprotectin level is i) maintained at about the same level as the patient's fecal calprotectin level at the beginning of the subsequent period or ii) decreased within a range bounded at the upper end by the patient's fecal calprotectin level at the beginning of the subsequent period and at the lower end at a level about 40% less than the patient's fecal calprotectin level at the beginning of the subsequent period. In some embodiments, during a subsequent period for administration of a subsequent dose of Obefazimod, or a pharmaceutically acceptable salt thereof, as provided in a method or in a use of the present invention, a patient's fecal calprotectin level is i) maintained at about the same level as the patient's fecal calprotectin level at the beginning of the subsequent period or ii) decreased within a range bounded at the upper end by the patient's fecal calprotectin level at the beginning of the subsequent period and at the lower end at a level about 30% less than the patient's fecal calprotectin level at the beginning of the subsequent period. In some embodiments, during a subsequent period for administration of a subsequent dose of Obefazimod, or a pharmaceutically acceptable salt thereof, as provided in a method or in a use of the present invention, a patient's fecal calprotectin level is i) maintained at about the same level as the patient's fecal calprotectin level at the beginning of the subsequent period or ii) decreased within a range bounded at the upper end by the patient's fecal calprotectin level at the beginning of the subsequent period and at the lower end at a level about 20% less than the patient's fecal calprotectin level at the beginning of the subsequent period. In some embodiments, during a subsequent period for administration of a subsequent dose of Obefazimod, or a pharmaceutically acceptable salt thereof, as provided in a method or in a use of the present invention, a patient's fecal calprotectin level is i) maintained at about the same level as the patient's fecal calprotectin level at the beginning of the subsequent period or ii) decreased within a range bounded at the upper end by the patient's fecal calprotectin level at the beginning of the subsequent period and at the lower end at a level about 10% less than the patient's fecal calprotectin level at the beginning of the subsequent period.

In some embodiments, the present invention provides a method for treating a patient with moderately to severely active ulcerative colitis, the method comprising administering to the patient Obefazimod, or a pharmaceutically acceptable salt thereof, at an initial dose of 50 mg once a day for an initial period of 8 weeks, followed by a subsequent dose of 25 mg once a day for a subsequent period of 44 weeks. In some embodiments, the present invention provides a therapeutically effective amount of Obefazimod, or a pharmaceutically acceptable salt thereof, for use in the treatment of moderately to severely active ulcerative colitis in a patient, at an initial dose of 50 mg once a day for an initial period of 8 weeks, followed by a subsequent dose of 25 mg once a day for a subsequent period of 44 weeks.

In some embodiments, the present invention provides a method for treating a patient with moderately to severely active ulcerative colitis, the method comprising administering to the patient Obefazimod, or a pharmaceutically acceptable salt thereof, at an initial dose of 50 mg once a day for an initial period of 8 weeks, followed by a subsequent dose of 25 mg once a day for a subsequent period of 44 weeks, wherein the patient has an inadequate response, no response, a loss of response, or an intolerance to conventional therapies and/or advanced therapies for treatment of moderately to severely active ulcerative colitis. In some embodiments, the present invention provides a therapeutically effective amount of Obefazimod, or a pharmaceutically acceptable salt thereof, for use in the treatment of moderately to severely active ulcerative colitis in a patient, at an initial dose of 50 mg once a day for an initial period of 8 weeks, followed by a subsequent dose of 25 mg once a day for a subsequent period of 44 weeks, wherein the patient has an inadequate response, no response, a loss of response, or an intolerance to conventional therapies and/or advanced therapies for treatment of moderately to severely active ulcerative colitis.

In some embodiments, a subsequent dose period is started immediately after (e.g., the next day) an initial dose period. In some embodiments, a subsequent dose period is started at a time after an initial dose period has ended.

In some embodiments, Obefazimod, or a pharmaceutically acceptable salt thereof, is amorphous. In some embodiments, Obefazimod, or a pharmaceutically acceptable salt thereof, is in crystal form. In some embodiments, a crystal form of Obefazimod is as described in WO2020127839, the content of which is incorporated herein by reference in its entirety.

In some embodiments, a crystalline form of Obefazimod is a free base form. In some embodiments, a crystalline form of Obefazimod is characterized by one or more x-ray powder diffractogram (XRPD) degree 2-Theta angles selected from 7.3, 14.6, 18.4, and 24.9 (each angle±0.2). In some embodiments, a crystalline form of Obefazimod is characterized by one or more x-ray powder diffractogram (XRPD) degree 2-Theta angles selected from 18.0, 24.2, 28.3, and 29.5 (each angle±0.2). In some embodiments, a crystalline form of Obefazimod is characterized by one or more x-ray powder diffractogram (XRPD) degree 2-Theta angles selected from 18.6, 22.3, 23.0, and 23.5 (each angle±0.2).

In some embodiments, a crystalline form of Obefazimod is characterized by one or more x-ray powder diffractogram (XRPD) degree 2-Theta angles selected from 7.3, 14.6, 23.5, and 28.4 (each angle±0.2). In some embodiments, a crystalline form of Obefazimod is characterized by one or more x-ray powder diffractogram (XRPD) degree 2-Theta angles selected from 12.1, 17.3, 18.4, 23.0, 24.2, 24.9, 27.4 and 29.1 (each angle±0.2). In some embodiments, a crystalline form of Obefazimod is characterized by one or more x-ray powder diffractogram (XRPD) degree 2-Theta angles selected from 13.7, 16.3, 16.9, 18.1, 22.4, and 29.6 (each angle±0.2).

In some embodiments, Obefazimod is in a powder form as described in WO2022200426, the content of which is incorporated herein by reference in its entirety. In some embodiments, an Obefazimod powder has a particle size distribution having a D50 value of not more than 80.0 μm, in particular of not more than 70.0 μm, and for example from 30.0 μm to 70.0 μm. In some embodiments, an Obefazimod powder has a particle size distribution having a D10 value of not more than 20.0 μm, in particular of not more than 15.0 μm, and for example from 1.0 to 15.0 μm. In some embodiments, an Obefazimod powder has a particle size distribution having a D90 of not more than 190.0 μm, in particular of not more than 180.0 μm, and for example from 80.0 μm to 180.0 μm.

As used herein D10, D50 and D90 are so-called percentile values. These are statistical parameters that can be read directly from the cumulative particle size distribution (PSD). They indicate the size below which 10%, 50% or 90% of all particles are found. In one embodiment, said PSD is determined by means of laser light diffraction. In another embodiment, said PSD is determined by means of a wet method as detailed in WO2022200426, the content of which is incorporated herein by reference in its entirety.

In some embodiments, a patient meets one or more of the inclusion criteria as described in Examples 1, 2, and 3 herein.

In some embodiments, a patient is at least 16 years old. In some embodiments, a patient is an adolescent and weighs at least 40 kg. In some embodiments, a patient is at least 18 years old.

In some embodiments, a patient has ulcerative colitis, which is confirmed by endoscopy and histology.

In some embodiments, a patient of ulcerative colitis has an active disease with a modified Mayo score (MMS) of at least 5. In some embodiments, a patient of ulcerative colitis has an active disease with a rectal bleeding subscore (RBS) of at least 1. In some embodiments, a patient of ulcerative colitis has an active disease with an endoscopy subscore (MES) of 2 or 3.

In some embodiments, a patient has an inadequate response, no response, a loss of response, or an intolerance to at least one of the following treatments: corticosteroids, immunosuppressant, biologic therapies, S1P receptor modulators and/or JAK inhibitors. In some embodiments, a patient has an inadequate response, no response, a loss of response, or an intolerance to Mesalamine (also known as 5-aminosalicylic acid, or 5-ASA).

In some embodiments, an inadequate response to corticosteroids (CS) is a CS resistance (i.e. signs and symptoms of persistently active disease despite current or prior course of oral prednisone/prednisolone≥40 mg/d for at least 2 weeks, or to budesonide≥9 mg/d for at least 2 weeks, or to beclomethasone≥5 mg/d for at least 2 weeks). In some embodiments, an inadequate response to corticosteroids (CS) is a CS dependence (i.e. failure to taper to ≤10 mg of prednisone/prednisolone, <9 mg/d of budesonide or <5 mg of beclomethasone or relapse occurring within 3 months after stopping CS).

In some embodiments, an intolerance to CS includes, but is not limited to, one of more of Cushing's syndrome, osteopenia/osteoporosis, hyperglycemia, insomnia, and infection.

In some embodiments, a patient having an inadequate response to immunosuppressants has signs and symptoms of persistently active disease despite azathioprine treatment at 1.5 to 2.5 mg/kg/day for at least 8 weeks, or mercaptopurine 0.5 to 1.5 mg/kg/day for at least 8 weeks, or methotrexate 12.5 mg to 15 mg/week for at least 8 weeks.

In some embodiments, an intolerance to immunosuppressant includes, but is not limited to, one of more of nausea, vomiting, abdominal pain, pancreatitis, liver enzyme abnormalities, lymphopenia, and infection.

In some embodiments, a patient having an inadequate response to biologics is a primary non responder to full induction course as in the USPI/SmPC of the following:
infliximab or biosimilars (≥5 mg/kg intravenously at 0, 2, and 6 weeks);
adalimumab or biosimilars (160 mg subcutaneous dose followed by 80 mg SC [or 80 mg SC dose in countries where this dosing regimen is allowed] followed by 40 mg SC dose at least 2 weeks apart);
golimumab (200 mg SC dose followed by 100 mg SC dose at least 2 weeks apart);
vedolizumab (300 mg IV at 0, 2, and 6 weeks); and/or
ustekinumab (one single IV using weight-based dosing −260 mg for subjects with body≤55 kg; 390 mg for subjects with body weight>55 kg and ≤85 kg; 520 mg for subjects with body weight>85 kg).

In some embodiments, an inadequate response to biologics is a loss of response, where there is a recurrence of symptoms during a maintenance course following primary response after a full induction course. In some embodiments, an inadequate response to biologics is an intolerance, which includes, but is not limited to, one or more of infusion-related reaction, serious opportunistic infection, and malignancies.

In some embodiments, a patient having an inadequate response to JAK inhibitors has signs and symptoms of persistently active disease despite at least one full induction course according to USPI/SmPC of tofacitinib (i.e., 8 weeks), or to at least one full induction course according to SmPC of filgotinib (i.e., 10 weeks), or to at least one full induction course according to USPI of upadacitinib (i.e., 8 weeks), or recurrence of symptoms during maintenance course. In some embodiments, an intolerance to JAK inhibitor treatment includes, but is not limited to, one or more of an increase of serious infection, malignancies, deep venous thrombosis (DVT), and a major adverse cardiac event (MACE).

In some embodiments, a patient having an inadequate response to S1P receptor modulators has signs and symptoms of persistently active disease despite at least one full induction course according to USPI/SmPC of ozanimod (i.e., at least 12 weeks) or recurrence of symptoms during maintenance course. In some embodiments, an intolerance to ozanimod includes, but is not limited to, one or more of serious infections, liver enzyme, cardiac abnormalities, and lymphopenia.

In some embodiments, a patient does not meet one or more of the exclusion criteria as described in Examples 1, 2, and 3 herein.

In some embodiments, a patient is not a patient with ulcerative colitis limited to an isolated proctitis (≤15 cm from anal verge).

In some embodiments, a patient is not a patient with primary sclerosing cholangitis or autoimmune hepatitis.

In some embodiments, a patient is not a patient, who has no response, a loss of response, or an intolerance to Mesalamine (also known as 5-aminosalicylic acid, or 5-ASA) therapy only.

In some embodiments, a patient is not a patient with CD or presence or history of fistula, indeterminate colitis, infectious/ischemic colitis or microscopic colitis (lymphocytic and collagenous colitis).

In some embodiments, a patient is not a patient having a history or current evidence of toxic megacolon, fulminant colitis, or bowel perforation.

In some embodiments, a patient is not a patient having a history of colon cancer, a past or current evidence of low grade or high-grade colonic dysplasia and/or adenomatous polyps that have not been completely removed.

In some embodiments, a patient is not a patient having a recent or planned bowel surgery or history of proctocolectomy or partial colectomy or current stoma.

In some embodiments, a patient is not a patient on antidiarrheals including those working on motility (e.g., loperamide, diphenoxylate with atropine, etc.).

In some embodiments, a patient is not a patient on probiotics (e.g., Culturelle® [Lactobacillus GG, i-Health, Inc.], Saccharomyces boulardii).

In some embodiments, a patient is not a patient with one or more of the following hematological and biochemical laboratory parameters:
Hemoglobin≤8.0 g dL⁻¹;
Absolute neutrophil count<750 mm⁻³;
Platelets<100,000 mm⁻³;
Creatinine clearance<60 mL·min⁻¹ (Cockcroft-Gault formula);
Total serum bilirubin>1.5×ULN; and
Alkaline phosphatase, aspartate aminotransferase (AST) and alanine aminotransferase (ALT)>2×ULN.

In some embodiments, a patient is not a patient with chronic or recurrent grade 3 or grade 4 infection within the last 2 months prior to Obefazimod treatment, or a history of opportunistic infection while not on immunosuppressive therapy.

In some embodiments, a patient is not a patient with herpes zoster reactivation within the last 2 months prior to Obefazimod treatment.

In some embodiments, a patient is not a patient with any active infection or any major episode of infection that required hospitalization or treatment with intravenous antibiotics within 1 month of Obefazimod treatment (excluding fungal infection of nail beds).

In some embodiments, a patient is not a patient with a positive assay or stool culture for pathogens (ova and parasite examination, bacteria) or positive test for Clostridium difficile toxin. In some embodiments, a patient is C. difficile positive.

In some embodiments, a patient is not a patient with HIV infection.

In some embodiments, a patient is not a patient having acute or chronic hepatitis B infection (positive for hepatitis B surface antigen [HbsAg], or negative for HbsAg and positive for anti-hepatitis B core antibody in conjunction with detectable HBV DNA, or detectable HBV DNA).

In some embodiments, a patient is not a patient having acute or chronic hepatitis C infection, as defined by positive for hepatitis C antibody. In some embodiments, a patient having acute or chronic hepatitis C infection is successfully treated and without recurrence≥1 year with no detectable HCV RNA before Obefazimod treatment.

In some embodiments, a patient is not a patient with active tuberculosis (TB) or untreated latent TB. In some embodiments, a patient is a patient with a positive or intermediate QuantiFERON test.

In some embodiments, a patient is not a patient with an uncontrolled ischemic heart disease and/or a history of congestive heart failure with New York Heart Association (NYHA) class 3 or 4 symptoms.

In some embodiments, a patient is not a patient with a family or personal history of congenital or acquired long QT syndrome, or with a marked baseline prolongation of QT/QTc interval (e.g., repeated demonstration of a QTc interval [Fridericia or Bazett correction]>450 milliseconds for male and >460 milliseconds for female).

In some embodiments, a patient is not a patient with a history of torsade de pointe (TdP).

In some embodiments, a patient is not a patient with acute or chronic of clinically relevant pulmonary, hepatic, pancreatic or renal functional abnormality, encephalopathy, neuropathy or unstable central nervous system pathology such as seizure disorder, or any other clinically significant medical problems. In some embodiments, a patient has treated autoimmune hypothyroidy and/or autoimmune diabetes.

In some embodiments, a patient is not a patient with a history or active malignancy. In some embodiments, a patient is a patient with a 5-year disease free survival.

In some embodiments, a patient is not a patient with serious illness requiring hospitalization within 4 weeks prior to Obefazimod treatment. In some embodiments, a patient is a patient with UC flare requiring hospitalization within 4 weeks prior to Obefazimod treatment.

In some embodiments, a patient is not a patient previously treated with Obefazimod.

In some embodiments, a patient is not illicit drug or alcohol abuse or dependence.

In some embodiments, a patient has not received live vaccine within 3 months prior to Obefazimod treatment.

In some embodiments, a patient has not received any investigational or non-registered product within 3 months or within 5 half-lives before Obefazimod treatment.

### EXEMPLIFICATION

Obefazimod, or a pharmaceutically acceptable salt thereof, or a pharmaceutical composition thereof, can be prepared by methods known to one skilled in the art, for example, as described in WO2010/143169, WO2012/080953, WO2016/009065, WO2016/009066, WO2020127839, and WO2022200426, the contents of each of which are incorporated herein by reference in their entireties.

### List of Abbreviations

| Abbreviation | |
|---|---|
| or Term | Definition |
| AE | Adverse event |
| AESI | Adverse event of special interest |
| ALT | Alanine aminotransferase |
| ANCOVA | Analysis of covariance |
| AST | Aspartate aminotransferase |
| BU | Bowel Urgency |
| CCL2/MCP1 | Chemokine for monocytes: Monocyte chemoattractant |
| | protein-1, MCP-1) |
| CD | Crohn's disease |
| CECT | Contrast-enhanced computed tomography |
| CFR | Code of Federal Regulations |
| CI | Confidence Interval |
| CMH | Cochran Mantel Haenszel |
| CPK | Creatine phosphokinase |
| CRA | Clinical Research Associate |
| CRO | Contract research organization |
| CRP | C Reactive Protein |
| CS | Corticosteroids |
| CT | Corneal Transparency |
| CTCAE | Common Terminology Criteria for Adverse Events |
| CTFG | Clinical trial facilitation group |
| CYP | Cytochrome P450 |
| DSS | Dextran Sodium Sulfate |
| DTP | Direct to Patient or Subject |
| DVT | Deep Venous Thrombosis |
| ECG | Electrocardiogram |
| eCRF | Electronic case report form |
| EDC | Electronic Data Capture |
| EIM | Extra Intestinal Manifestations |
| e-Diary | Electronic diary |
| EM | Hospital Emergency Room |
| EoS | End of Study |
| EoT | End of Treatment |
| EQ-5D-5L or | Euro Quality of Life - 5 Dimensions questionnaire |
| EuroQoL-5D | |
| FACIT-Fatigue | Functional Assessment of Chronic Illness |
| | Therapy-Fatigue |
| FC | Fecal Calprotectin |
| FDA | Food and Drug Administration |
| GCP | Good Clinical Practice |
| GGT | Gamma-glutamyl transferase |
| GLS | Global longitudinal strain |
| GMP | Good Manufacturing Practice |
| HBsAg | Hepatitis B surface antigen |
| HBV | Hepatitis B virus |
| HCV | Hepatitis C virus |
| HEMI | Histologic-endoscopic mucosal improvement |
| HEMR | Histologic-endoscopic mucosal remission |
| HIV | Human immunodeficiency virus |
| hsCRP | High sensitive C reactive protein |
| HuMDM | Human monocyte-derived macrophages |
| IBD | Inflammatory bowel disease |
| IBDQ | Inflammatory bowel disease questionnaire |
| ICF | Informed consent form |
| ICH | International Council for Harmonization |
| ICVSC | Independent Cardiovascular Safety Committee |
| IDMC | Independent Data Monitoring Committee |
| IEC | Independent Ethics Committee |
| IL | Interleukin |
| IMID | Immune Modulated Inflammatory Disease |
| IMP | Investigational Medicinal Product |
| INR | International normalized ratio |
| IRB | Institutional review board |
| ITT | Intent-to-treat |
| IS | Immunosuppressants |
| IWRS | Interactive web response system |
| JAK | Janus kinase |
| LFT | Liver function test |
| LVEF | Left ventricular ejection fraction |
| MACE | Major Adverse Cardiac Event |
| MedDRA | Medical Dictionary for Regulatory Activities |
| MES | MAYO Endoscopic Sub-score |
| miR | Micro-RNA |
| MMRM | Mixed model for repeated measures |
| MMS | Modified Mayo Score |
| NBM | Noocturnal Bowel Movements |
| NRI | Non-Responder Imputation |
| NOAEL | No-observed-adverse-effect level |
| NRS | Numeric Rating Scale |
| NT-proBNP | N-terminal pro-B-type natriuretic peptide |
| OAT | Organic anion transporter |
| OC | Observed Cases |
| OR | Odds Ratio |
| PBD | Prevention of Blindness and Deafness |
| PBL | Prevention of Blindness |
| PBMC | Peripheral blood mononucleated cells |
| PK | Pharmacokinetics |
| PMI | Placebo Multiple imputation |
| pMMS | Partial Modified Mayo Score |
| popPK | Population pharmacokinetic |
| PRO | Subject-Reported Outcome |
| QD | Once daily |
| RA | Rheumatoid arthritis |
| RBS | Rectal Bleeding Sub-Score |
| RCV | Relapse Confirmatory Visit |
| RHI | Robarts Histopathology Index |
| RNA | Ribonucleic acid |
| S1P | Sphingosine 1-phosphate |
| SAE | Serious adverse event |
| SAP | Statistical analysis plan |
| SAR | Serious adverse reaction |
| SD | Standard deviation |
| SF | Stool Frequency Sub-Score |
| SEM | Standard error of the mean |
| SmPC | Summary of Product Characteristics |
| SoA | Schedule of Assessments |
| SOC | System organ class |
| SSC | Study Steering Committee |
| STAT3 | Signal transducer and activator of transcription 3 |
| SUSAR | Suspected Unexpected Serious Adverse Reaction |
| TB | Tuberculosis |
| TEAE | Treatment emergent adverse event |
| Th | T helper |
| TMF | Trial Master File |
| TNF | Tumor necrosis factor |
| UC | Ulcerative colitis |
| UGT | UDP-glucuronosyltransferase |
| ULN | Upper limit of normal |
| USPI | United States Prescribing Information |
| VA | Visual acuity |
| VAS | Visual analogue scale |
| WHO | World Health Organization |
| WOCBP | Women of childbearing potential |
| WPAI | Work Productivity and Activity Impairment |
| | questionnaire |
| β-hCG | βeta-human chorionic gonadotropin |

### Example 1. A Randomized, Double-Blind, Placebo-Controlled, Multicenter Phase III Study to Evaluate the Efficacy and Safety of ABX464 Once Daily for Induction Treatment in Subjects with Moderately to Severely Active Ulcerative Colitis (ABX464-105)

### Study Specific Definitions

For the purpose of the study, the following definitions will be applied:
Modified Mayo Score (MMS) is defined as 3-component Mayo Score: Rectal bleeding subscore (RBS), Stool Frequency subscore (SFS) and Mayo Endoscopic subscore (MES)
Partial Modified Mayo Score (pMMS) is defined as combination of RBS and SFS
Total Mayo Score is defined as combination of RBS, SFS, MES and physician global assessment (PGA)
Clinical remission per MMS is defined as SFS=0 or 1 and RBS=0, and MES=0 or 1
Clinical response per MMS is defined as a reduction from baseline in MMS≥2 points and ≥30% associated to a reduction≥1 point in RBS and/or RBS=0 or 1
Partial Clinical response per pMMS is defined as a reduction from baseline in pMMS≥1 points and ≥30% associated to a reduction≥1 point in RBS and/or RBS=0 or 1
Symptomatic remission is defined as SFS=0 or 1 (with a 1-point reduction from baseline) and RBS=0
Endoscopic improvement is defined as MES=0 or 1
Endoscopic remission defined as a MES=0
Histologic improvement is defined as Geboes score≤3.1 [or ≤4 per Robarts Histopathology Index (RHI)]
Histologic remission is defined as Geboes score≤2A [or ≤3 per Robarts Histopathology Index (RHI)]
Histologic-endoscopic mucosal improvement (HEMI) is defined as endoscopic improvement associated with histologic improvement
Histologic-endoscopic mucosal remission (HEMR) is defined as endoscopic remission associated with histologic remission.
Conventional therapies include 5-ASA, corticosteroids (CS), immunosuppressants (IS)
Advanced therapies (AT) include biologics [anti-TNF i.e. adalimumab, infliximab, golimumab, anti-integrins i.e. vedolizumab, anti-IL23 i.e. ustekinumab], S1P receptor modulators (i.e. ozanimod), JAK inhibitors (i.e. tofacitinib, filgotinib, upadacitinib) and potential new approved drugs.
Nocturnal Bowel Movement (NBM) is defined as one or more sleep interruption due to bowel movement.
Bowel Urgency (BU) is defined as a sudden, almost uncontrollable need to go to the toilets due to bowel movements.
Extra Intestinal Manifestations (EIM) are defined as: ophthalmological manifestations (uveitis), rheumatological manifestations (axial spondyloarthritis, peripheral spondyloarthritis, arthralgia) or dermatological manifestations (psoriasis, pyoderma gangrenosum, erythema nodosum).

### Study Objectives and Endpoints

### Primary Objective and Endpoint:

The primary objective is to compare the efficacy of ABX464 versus placebo on clinical remission.

Proportion of subjects who achieve clinical remission per Modified Mayo Score at week 8.

### Key Secondary Efficacy Objectives and Endpoints

The key secondary efficacy objectives and endpoints are:
To compare the efficacy of ABX464 versus placebo on endoscopic improvement.
   Proportion of subjects who achieve endoscopic improvement at week 8.
To compare the efficacy of ABX464 versus placebo on clinical response as per MMS.
   Proportion of subjects who achieve clinical response per MMS at week 8.
To compare the efficacy of ABX464 versus placebo on symptomatic remission.
   Proportion of subjects with symptomatic remission at week 8.
To compare the efficacy of ABX464 versus placebo on histologic-endoscopic mucosal improvement (HEMI).
   Proportion of subjects with HEMI per Geboes at week 8.

### Other Secondary Objectives and Efficacy Endpoints:

To compare the efficacy of ABX464 versus placebo on partial clinical response as per pMMS.
   Proportion of subjects with partial clinical response over time.
To compare the efficacy of ABX464 versus placebo on RBS.
   Proportion of subjects with no rectal bleeding over time.
To compare the efficacy of ABX464 versus placebo on SFS.
   Proportion of subjects with SFS=0 or 1 over time.
To compare the efficacy of ABX464 versus placebo on Bowel Urgency (BU).
   Proportion of subjects with BU=0 over time.
To compare the efficacy of ABX464 versus placebo on Fecal Calprotectin (FCP).
   Change from baseline in FCP over time.
   Proportion of subjects with fecal calprotectin below 150 μg/g over time.
To compare the efficacy of ABX464 versus placebo on Fatigue Numeric Rating Scales (NRS).
   Change from baseline in the fatigue NRS over time.
To compare the efficacy of ABX464 versus placebo on FACIT-Fatigue questionnaire.
   Change from baseline in the FACIT-Fatigue at week 8.
To compare the efficacy of ABX464 versus placebo on histologic improvement.
   Proportion of subjects who achieve histologic improvement per Geboes (and RHI) scoring at week 8.
To compare the efficacy of ABX464 versus placebo on endoscopic remission.
   Proportion of subjects who achieve endoscopic remission at week 8.
To compare the efficacy of ABX464 versus placebo on histologic endoscopic mucosal remission (HEMR).
   Proportion of subjects with HEMR Geboes (and RHI) scoring at week 8.
To compare the efficacy of ABX464 versus placebo on histologic remission.
   Proportion of subjects who achieve histologic remission Geboes (and RHI) scoring at week 8.
To compare the efficacy of ABX464 versus placebo on IBDQ.
   Change from Baseline in IBDQ total score and subscores at week 8.
   Proportion of subjects with IBDQ response (increase of IBDQ≥16 from Baseline) at week 8.
To compare the efficacy of ABX464 versus placebo on nocturnal bowel movement (NBM).
   Proportion of subjects with no longer NBM at week 8 in the sub-population of subjects with NBM at baseline.
To compare the efficacy of ABX464 versus placebo on Extra Intestinal Manifestations (EIMs).
   Proportion of subjects with EIMs at week 8 in the subpopulation of subjects having EIM at baseline.
To compare the efficacy of ABX464 versus placebo on symptomatic remission.
   Proportion of subjects with symptomatic remission over time.
To compare the efficacy of ABX464 versus placebo on EQ-5D-5L.
   Change from baseline in EQ-5D-5L score over time.
To compare the efficacy of ABX464 versus placebo on hsCRP.
   Change from baseline in hsCRP over time.
To compare the efficacy of ABX464 versus placebo in WPAI.
   Change from baseline in WPAI score at week 8.
To compare the efficacy of ABX464 versus placebo on any UC related Emergency Room (ER) visits, hospitalizations and/or surgery.
   Proportion of subjects with ulcerative colitis related visits to ER, hospitalizations and/or surgery over time.
To compare the effect of ABX464 versus placebo on Total Mayo Score.
   Change from baseline in Total Mayo Score at week 8.
To compare the effect of ABX464 versus placebo on miR-124 expression in tissue and in blood.
   Change from baseline in miRNA-124 expression in rectal/sigmoidal biopsies at week 8 and in blood over time.
To compare the effect of ABX464 versus placebo on proinflammatory cytokine plasma concentrations.
   Change from baseline in proinflammatory cytokine plasma concentrations over time.
To assess the pharmacokinetic (PK) parameters of ABX464 and its main active metabolite ABX464-N-Glu after oral administration.
   Serum concentrations of ABX464 and ABX464-N-Glucuronide at baseline, week 4 and week 8 (at specified time points depending on the group allocated via IWRS).

### Safety Objective and Endpoints:

To compare ABX464 safety profile versus placebo during induction:
Incidence of all treatment-emergent adverse events (TEAEs), causally related TEAEs, all serious adverse events (SAE) and causally related SAEs classified by severity.
Incidence of adverse events leading to investigational medicinal product (IMP) discontinuation and study discontinuation.
Incidence of adverse events of special interest (AESIs).
Incidence of clinically significant laboratory abnormalities.
Incidence of clinically significant abnormalities regarding vital signs and ECG

### Cardiac Safety Sub-Study: Objectives and Endpoints

To assess the safety of ABX464 versus placebo on cardiac function assessed by echocardiography at week 8.

### Study Design and Methodology

This is a multicenter, randomized, placebo controlled study to evaluate the efficacy and safety of ABX464 given at 25 or 50 mg QD in inducing clinical remission in subjects with moderately to severely active ulcerative colitis who have inadequate response, no response, a loss of response, or an intolerance to either conventional therapies [corticosteroids, immunosuppressant (i.e. azathioprine, 6-mercaptopurine, methotrexate)] and/or advanced therapies [biologics (TNF inhibitors, anti-integrins, anti-IL-23), and/or S1P receptor modulators, and/or JAK inhibitors].

As shown below, this study consists of a screening period of up to 28 days, an 8-week induction period followed by 28-day follow up period. Subjects who complete the 8-week induction will be given the opportunity to take part in the maintenance study (i.e. ABX464-107) either:
In the randomized double-blind placebo-controlled study part [part 1; placebo, 25 mg, 50 mg] for clinical responders at week 8; and
In the open label arms study part [part 2; 25 mg or 50 mg] for non-clinical responders at week 8.

Approximately 612 subjects, aged at least 16 years, will be randomized in this study.

On Day 1, eligible subjects will be randomized and allocated into three treatment arms according to ratio 1:1:2 as follows:
Placebo: 153 subjects
ABX464—Daily dose 25 mg QD: 153 subjects
ABX464—Daily dose 50 mg QD: 306 subjects

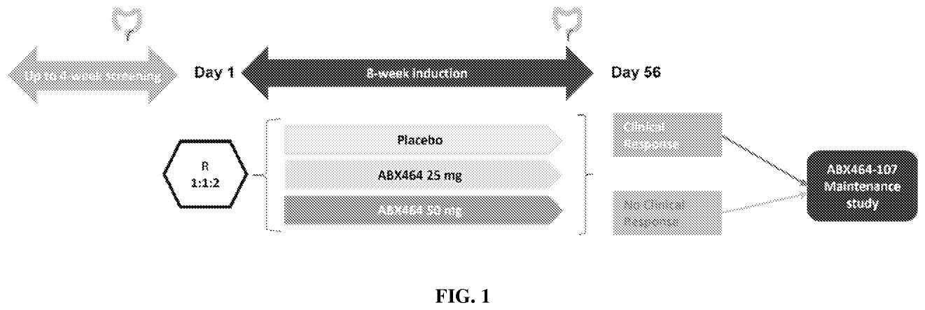

### Study Design (See FIG. 1)

Randomization will be stratified according to the following factors:
Subjects with inadequate response to advanced therapies [AT-IR subjects](no response, loss of response or intolerance to biologics, S1P receptor modulators, JAK inhibitors) (Yes/No)
Subjects with concomitant corticosteroids at baseline (Yes/No)

Approximately 60% of the enrolled population should be subjects with inadequate response to Advanced Therapies (biologics, S1P receptor modulators, JAK inhibitors). In addition, percentage of subjects who have failed to respond to JAK inhibitors should be limited to 15% of subjects with inadequate response to Advanced Therapies.

Subjects with previous exposure to Advanced Therapies without a documented inadequate response will be considered as non-AT-IR subjects.

Following Day 1 (randomization day), subjects will come at the investigational site at Day 28 (Week 4) and Day 56 (Week 8) according to the study schedule of assessments.

At Day 56, subjects will be given the choice to either take part in the maintenance study (ABX464-107) or to end their participation in the present induction study by completing the study. For the latter option, subjects will return for an end of study visit 28 days after the last study drug intake. However, subjects who are eligible for the maintenance study should be encouraged to enter the maintenance study.

Subjects will be evaluated for safety and efficacy throughout the induction study.

During the screening period, a full colonoscopy will be performed for all subjects. At week 8, a flexible sigmoidoscopy is only required. At screening and week 8, during both endoscopic procedures, biopsies at the most severely affected area will be performed. Endoscopies will be centrally read. Sigmoidoscopy might be substituted by a full colonoscopy if requested by national medical practices or in case of proximal disease involvement. Biopsies will be centrally analyzed.

e-Diaries will be used to collect stool frequency, rectal bleedings, nocturnal bowel movements, bowel urgency and fatigue NRS on a daily basis, as well as the study drug intake time.

PK samples will be collected in all subjects. Subjects will be allocated into two different PK groups via IWRS. At each time point collection (D1, D28, D56) samples will be taken as follows:
for group 1: pre-dose, 0.5, 1.5, 3 h (and 6 h and/or 10 h if possible) post-dose
for group 2: pre-dose, 1, 2, 4 h (and 8 h and/or 12 h if possible) post-dose

In addition, a cardiac safety sub-study consisting in a cardiac ultrasound performed at baseline and Week 8 (and potentially over the maintenance study if the subject takes part) will be performed in selected sites with appropriate equipment and resources. Echocardiograms will be centrally read and reviewed by an Independent Cardiovascular Safety Committee. 70 subjects or more per treatment arm (coming from both induction studies) will be part of the sub-study cardiac assessment. In addition, cardiac biomarkers will be tested through induction study in all participants.

Statistical considerations and methods are specified in the SAP.

### Inclusion Criteria:

A subject will be eligible to participate in this study if ALL the following criteria are met:
1. Men or women at least 16 years old; Adolescent subjects will only be enrolled if approved by the country regulatory/health authority. If these approvals have not been granted, only subjects≥18 years old will be enrolled. To be eligible, adolescent subjects must weight≥40 kg and meet the definition of Tanner Stage 5 at the screening visit.
2. Subjects must understand, sign and date the written voluntary informed consent form at the visit prior to any protocol-specific procedures. For under-aged subjects, national requirements regarding consent should also be met.
3. Documented diagnosis of UC confirmed by endoscopy and histology. Should histology results not be available at screening, results from biopsies taken at screening may be used.
4. Active disease defined by modified Mayo score (MMS)≥5 with rectal bleeding subscore (RBS)≥1 and endoscopy subscore (MES) of 2 or 3 (confirmed by central reader).
5. Subjects with documented inadequate response (defined as lack of response or loss of response or intolerance) to at least one of the following treatments: corticosteroids, immunosuppressant, biologic therapies, S1P receptor modulators and/or JAK inhibitors and/or new drugs approved during the study (note: failure to only 5-ASA is not accepted).
   a. Inadequate response to corticosteroids (CS) is defined as either a CS resistance (i.e. signs and symptoms of persistently active disease despite current or prior course of oral prednisone/prednisolone≥40 mg/d for at least 2 weeks, or to budesonide≥9 mg/d for at least 2 weeks, or to beclomethasone≥5 mg/d for at least 2 weeks) or a CS dependence (i.e. failure to taper to ≤10 mg of prednisone/prednisolone, <9 mg/d of budesonide or <5 mg of beclomethasone or relapse occurring within 3 months after stopping CS).
      Intolerance to CS includes (but not limited to) Cushing's syndrome, osteopenia/osteoporosis, hyperglycemia, insomnia, infection.
   b. Inadequate response to immunosuppressants is defined as signs and symptoms of persistently active disease despite azathioprine treatment at 1.5 to 2.5 mg/kg/day for at least 8 weeks, or mercaptopurine 0.5 to 1.5 mg/kg/day for at least 8 weeks, or methotrexate 12.5 mg to 15 mg/week for at least 8 weeks
      Intolerance to immunosuppressant includes (but not limited to) nausea/vomiting, abdominal pain, pancreatitis, liver enzyme abnormalities, lymphopenia, infection.
   c. Inadequate response to biologics is defined as:
      Primary non responders to full induction course as in the USPI/SmPC of the following:
         infliximab or biosimilars (≥5 mg/kg intravenously at 0, 2, and 6 weeks),
         adalimumab or biosimilars (160 mg subcutaneous dose followed by 80 mg SC [or 80 mg SC dose in countries where this dosing regimen is allowed] followed by 40 mg SC dose at least 2 weeks apart),
         golimumab (200 mg SC dose followed by 100 mg SC dose at least 2 weeks apart),
         vedolizumab (300 mg IV at 0, 2, and 6 weeks),
         ustekinumab (one single IV using weight-based dosing −260 mg for subjects with body≤55 kg; 390 mg for subjects with body weight>55 kg and ≤85 kg; 520 mg for subjects with body weight>85 kg),
      Loss of response is defined as recurrence of symptoms during maintenance course following primary response after full induction course (discontinuation despite clinical benefit does not qualify)
      Intolerance includes (but not limited to) infusion-related reaction, serious opportunistic infection, malignancies.
   d. Inadequate response to JAK inhibitors is defined as signs and symptoms of persistently active disease despite at least one full induction course according to USPI/SmPC of tofacitinib (i.e., 8 weeks), or to at least one full induction course according to SmPC of filgotinib (i.e., 10 weeks), or to at least one full induction course according to USPI of upadacitinib (i.e., 8 weeks), or recurrence of symptoms during maintenance course.
      Intolerance to JAK inhibitor treatment that includes (but not limited to) increase of serious infection, malignancies, deep venous thrombosis (DVT), or major adverse cardiac event (MACE).
   e. Inadequate response to S1P receptor modulators is defined as signs and symptoms of persistently active disease despite at least one full induction course according to USPI/SmPC of ozanimod (i.e., at least 12 weeks) or recurrence of symptoms during maintenance course. Intolerance to ozanimod that includes (but not limited to) serious infections, liver enzyme, cardiac abnormalities, lymphopenia.
   f. Inadequate response or intolerance to any new drug approved for moderate to severe active UC during the course of the study defined according to USPI/SmPC.
6. Women of childbearing potential (WOCBP) subjects and male subjects with WOCBP partner must agree to use highly effective contraception methods.
7. Subjects able and willing to comply with study visits and procedures as per protocol.
8. Subjects should be affiliated to a health insurance policy whenever required by a participating country or state.

### Exclusion Criteria:

Subjects who meet ANY of the following exclusion criteria will be excluded from the study:
1. Subjects with ulcerative colitis limited to an isolated proctitis (≤15 cm from anal verge).
2. Subjects with primary sclerosing cholangitis or autoimmune hepatitis.
3. Subjects who have failed on 5-ASA therapy only.
4. Subjects with CD or presence or history of fistula, indeterminate colitis, infectious/ischemic colitis or microscopic colitis (lymphocytic and collagenous colitis).
5. History or current evidence of toxic megacolon, fulminant colitis, bowel perforation.
6. History of colon cancer, past or current evidence of low grade or high-grade colonic dysplasia and/or adenomatous polyps that have not been completely removed.
7. Recent or planned bowel surgery or history of proctocolectomy or partial colectomy or current stoma.
8. Subjects on antidiarrheals including those working on motility (e.g., loperamide, diphenoxylate with atropine, etc.).
9. Subjects on probiotics (e.g., Culturelle® [Lactobacillus GG, i-Health, Inc.], Saccharomyces boulardii).
10. Subjects who do not meet the washout period requirements prior to the screening endoscopy.
11. Subjects with the following hematological and biochemical laboratory parameters obtained during the screening period:
   Hemoglobin≤8.0 g dL⁻¹
   Absolute neutrophil count<750 mm⁻³
   Platelets<100,000 mm⁻³
   Creatinine clearance<60 mL·min⁻¹ (Cockcroft-Gault formula)
   Total serum bilirubin>1.5×ULN
   Alkaline phosphatase, aspartate aminotransferase (AST) and alanine aminotransferase (ALT)>2×ULN
12. Subjects with the following conditions (infection):
   Subjects with chronic or recurrent grade 3 or grade 4 infection within the last 2 months prior to screening or a history of opportunistic infection while not on immunosuppressive therapy.
   Herpes zoster reactivation within the last 2 months prior to screening.
   Subjects with active infection at screening or any major episode of infection that required hospitalization or treatment with intravenous antibiotics within 1 month of screening or during screening. Fungal infection of nail beds is allowed.
   Positive assay or stool culture for pathogens (ova and parasite examination, bacteria) or positive test for Clostridium difficile toxin at screening. If C. difficile is positive, subject may be treated and retested ≥2 weeks after completing treatment.
   Subject with HIV infection.
   Subjects having acute or chronic hepatitis B infection at screening (positive for hepatitis B surface antigen [HbsAg], or negative for HbsAg and positive for anti-hepatitis B core antibody in conjunction with detectable HBV DNA, or detectable HBV DNA).
   Subjects having acute or chronic hepatitis C infection at screening as defined by positive for hepatitis C antibody (subjects successfully treated and without recurrence≥1 year with no detectable HCV RNA are eligible).
   Active tuberculosis (TB) or untreated latent TB are ruled out.
13. Subjects with an uncontrolled ischemic heart disease and/or a history of congestive heart failure with New York Heart Association (NYHA) class 3 or 4 symptoms.
14. Subjects with a family or personal history of congenital or acquired long QT syndrome, or subjects with a marked baseline prolongation of QT/QTc interval (e.g., repeated demonstration of a QTc interval [Fridericia or Bazett correction]>450 milliseconds for male and >460 milliseconds for female).
15. Subjects with a history of torsade de pointe (TdP).
16. Acute or chronic of clinically relevant pulmonary, hepatic, pancreatic or renal functional abnormality, encephalopathy, neuropathy or unstable central nervous system pathology such as seizure disorder, or any other clinically significant medical problems as determined by physical examination and/or laboratory screening tests and/or medical history (note: treated autoimmune hypothyroidy and autoimmune diabetes are allowed).
17. History or active malignancy (subjects with a 5-year disease free survival are eligible).
18. Serious illness requiring hospitalization within 4 weeks prior to screening (except UC flare).
19. Subjects previously treated with ABX464.
20. WOCBP subject and WOCBP partner of male subject who are pregnant at screening, intend or are planning to become pregnant during the study duration, and breast-feeding women.
21. Illicit drug or alcohol abuse or dependence.
22. Subjects who received live vaccine within 3 months prior to screening and/or who's planning to receive such a vaccine during the study duration.
23. Use of any investigational or non-registered product within 3 months or within 5 half-lives preceding baseline, whichever is longer, and during the study.
24. Any condition, which in the opinion of the investigator, could compromise the subject's safety or adherence to the study protocol.

### IMP, Dose, and Mode of Administration

Depending on the treatment arm allocation, ABX464/Placebo (capsules) 50 mg and 25 mg are administered once daily in the morning with food for 8 weeks during the induction phase.

### Medications

All previous UC medications on which failure of treatment has occurred will be recorded into the eCRF without time limitation for advanced therapies and over the past 2 years for conventional treatments. Previous and current medications will be reviewed to check the use of potentially prohibited medications and ensure appropriate washout periods are respected. All concomitant medications will be recorded into the eCRF.

### Allowed Concomitant UC Medications

For the treatment of UC (provided stable dose for at least 2 weeks prior to the screening endoscopy):
Corticosteroids: prednisone or prednisone equivalent≤15 mg/day; beclomethasone dipropionate (≤5 mg/day) or budesonide MMX (≤9 mg/day).
Oral 5-ASA.

### Prohibited Previous and Concomitant Medications

The following drugs are prohibited from enrollment, through the study, until study completion. For some of them used prior to subjects' enrollment a strict washout period may apply. Sites are allowed to assay drug levels during the study period to shorten the washout periods.

Ustekinumab must be stopped at least 12 weeks prior to Screening endoscopy.

Infliximab, adalimumab, golimumab, vedolizumab and ozanimod must be stopped at least 8 weeks prior to Screening endoscopy.

Cyclosporine, tacrolimus, mycophenolate mofetil, or thalidomide must be stopped at least 4 weeks prior to Screening endoscopy.

Parenteral or enteral nutrition must be stopped at least 3 weeks prior to screening endoscopy.

Tofacitinib, filgotinib, upadacitinib must be stopped at least 2 weeks prior to Screening endoscopy.

Rectal aminosalicylates or corticosteroids, other enemas/suppositories (other than required for endoscopy), must be stopped at least 2 weeks prior to the Screening endoscopy.

Azathioprine or 6-mercaptopurine or methotrexate must be stopped at least 2 weeks prior to Screening endoscopy.

Intravenous corticosteroids must be stopped at least 2 weeks prior to Screening endoscopy.

UC-related antibiotics that have been discontinued must be stopped at least 2 weeks of Screening endoscopy.

Topical (i.e. rectal) corticosteroids and/or 5-ASA must be stopped at least 2 weeks prior to Screening endoscopy

Antidiarrheals including those working on motility (e.g., loperamide, diphenoxylate with atropine) must be stopped at least 2 weeks prior to Screening endoscopy. Any use of antidiarrheals for acute and severe diarrhea must be documented in the dedicated eCRF.

Probiotics, fish oil, fecal transplantation must be stopped at least 2 weeks prior to Screening endoscopy.

Traditional Chinese medicine must be stopped at least 2 weeks prior to Screening endoscopy.

Non-steroidal anti-inflammatory drugs (NSAIDs) must be stopped at least 1 week prior to Screening endoscopy and during the study (except topical NSAIDs and the use of low dose aspirin for cardiovascular [CV] protection or short course [less than 7 days] of NSAIDs for treatment of adverse event).

Vaccination with live components is prohibited during the course of the study and up to 8 weeks after the last ABX464 dosing (of note, COVID vaccination is allowed with the exception of live-attenuated vaccine).

Drugs that inhibit or induce CYP1A2.

Drugs that inhibit UDP-glucuronosyltransferase (UGT) IA9 activity and inhibitors of organic anion transporter (OAT) P1B1/P1B3 transporters.

Natalizumab: no previous exposure allowed.

Use of any investigational or non-registered product is prohibited within 3 months or within 5 half-lives preceding baseline, whichever is longer and during the study.

### Premature Trial Discontinuation

Subject's premature trial discontinuation must occur for the following reasons:
An AE or an intercurrent condition that precludes continuation of treatment.
Specifically, an increase≥3.0×ULN in liver transaminases (AST and/or ALT) or an increase≥2.0×ULN in alkaline phosphatase or in total bilirubin requires close observation with repeating liver enzymes and serum bilirubin tests twice weekly and clinical investigation to understand the etiology of this elevation. Frequency of retesting can decrease to once a month if abnormality stabilizes after the initial 2 weeks of follow-up and if the subject is asymptomatic. Discontinuation of the study treatment should occur if:
   ALT or AST>8×ULN.
   ALT or AST>5×ULN for more than 2 weeks.
   ALT or AST>3×ULN and total bilirubin>2×ULN or INR>1.5.
   ALT or AST>3×ULN with appearance of fatigue, nausea, vomiting, right upper quadrant pain or tenderness, fever, rash, and/or eosinophilia (>5%).
Severe (grade 3 or higher) infection, a severe (grade 3 or higher) opportunistic infection, or sepsis.
Any medically significant abnormal laboratory results, including new onset anemia (defined as a hemoglobin decrease>2 g/dL from baseline or hemoglobin<8 g/dL)—note: transient abnormal values, and/or abnormal values related to an existing condition that doesn't constitute a medically significant worsening will not be a criterion for discontinuation.
Onset of acute pancreatitis (as defined by Atlanta criteria).
Any cardiac AESI or any cardiac condition diagnosed during the course of the study and assessed by the Independent Cardiovascular Safety Committee as changing the risk/benefit balance for the subject.
Any malignancies, including dysplastic skin lesions.
Any relevant toxicity or negative change in the risk/benefit assessment leading to an unacceptable risk for the subject (i.e., occurrence of AEs which character, severity or frequency is new in comparison to the existing risk profile), or any data deriving from other clinical trials or toxicological studies which negatively influence the risk/benefit assessment.
Pregnancy.
Investigator's decision.
Withdrawal of consent.
Administrative reasons from Sponsor (study level).

In case of premature discontinuation, subjects will be invited to perform an End Of Treatment (EOT) visit as per study scheduled of assessment.

### Subject Follow-Up

For completed subjects, the last dose of the induction study will be taken the day before the last visit (D56±2 days).

Subjects eligible for maintenance will be encouraged to take part in the ABX464-107 maintenance. The first treatment dose taken in the frame of the maintenance study will be taken after the study specific informed consent is signed.

Subjects not willing to take part in the maintenance study will come for a last safety follow-up visit and will be then followed as per the site standard of care after this last study visit.

### Sample Size Calculation

A total of 612 subjects (153:153:306) will allow the detection of a difference of at least 13% in the clinical remission rate at W8 between ABX464 50 mg (and 25 mg) and placebo with a statistical power of at least 90%. This calculation, based on a Chi-square test with a type I error rate of 5% (two-sided), assumes a placebo clinical remission rate of 8%. The reason for having the 1:1:2 allocation is to generate a sufficient number of subjects on ABX464 50 mg with clinical response and to be enrolled into the maintenance study.

Sample size calculations was performed with nQuery, version 7.

### Statistical Methods

### Efficacy:

The primary endpoint, clinical remission (based on the modified Mayo Scoring system, is defined as SFS=0 or 1 and no greater than baseline and RBS=0 and endoscopy subscore=0 or 1 (friability is scored as 2), will be analyzed using a Cochran Mantel Haenszel test (CMH) test adjusting for AT-IR (yes/no) and baseline corticosteroid treatment as stratification factors, using the ITT population.

Comparisons on the primary endpoint will be analyzed in sequence (clinical remission rate at Week 8 between the 50 mg dose group vs placebo and then clinical remission rate at Week 8 between the 25 mg dose group vs placebo).

If comparisons on primary endpoint for each dose are statistically significant (0.05 level, 2-sided situation), then, this testing procedure continued through each of the key secondary endpoints in the prespecified order until an endpoint failed to reach statistical significance (0.05 level, 2-sided situation), after which all subsequent key secondary endpoints would be considered exploratory.

Intercurrent events considered for primary estimand and their corresponding management are: premature discontinuation of study drug (composite policy), prohibited change in UC medications (composite variable strategy-NRI), premature discontinuation from study (composite variable strategy-NRI).

Sensitivity analyses, addressing estimand strategies together with the corresponding estimands will be performed.

### Safety:

AEs will be tabulated (counts and percentages) by group. All AEs will be listed, and the data will be tabulated by body system/organ class. AE tabulations will include all TEAEs, which will be further classified by severity and relationship to treatment and dose level.

Clinical laboratory parameters, vital signs, ECG will be summarized by using descriptive statistics (n, mean, SD, SEM, median, minimum and maximum). The number of subjects with at least one abnormal value will be tabulated (counts and percentages) for each parameter in summary shift tables, by group and dose.

### Pharmacokinetics:

A popPK model will be constructed for analysis of PK data.

### End of Trial Definition

If the last subject takes part to the maintenance study: End of Trial will be based on the last visit of the last remaining subject in the induction study.

If the last subject will not take part in the maintenance study, End of Trial will be the date of the last follow-up of the last remaining participant. In case of last subject is declared lost to follow-up, the date of the last follow-up or the date of the last contact attempt (whichever occurs later) should be considered.

### Example 2. A Randomized, Double-Blind, Placebo-Controlled, Multicenter Phase III Study to Evaluate the Efficacy and Safety of ABX464 Once Daily for Induction Treatment in Subjects with Moderately to Severely Active Ulcerative Colitis (ABX464-106)

### Study Specific Definitions

For the purpose of the study, the following definitions will be applied:
Modified Mayo Score (MMS) is defined as 3-component Mayo Score: Rectal bleeding subscore (RBS), Stool Frequency subscore (SFS) and Mayo Endoscopic subscore (MES)
Partial Modified Mayo Score (pMMS) is defined as combination of RBS and SFS
Total Mayo Score is defined as combination of RBS, SFS, MES and physician global assessment (PGA)
Clinical remission per MMS is defined as SFS=0 or 1 and RBS=0, and MES=0 or 1
Clinical response per MMS is defined as a reduction from baseline in MMS≥2 points and ≥30% associated to a reduction≥1 point in RBS and/or RBS=0 or 1
Partial Clinical response per pMMS is defined as a reduction from baseline in pMMS≥1 points and ≥30% associated to a reduction≥1 point in RBS and/or RBS=0 or 1
Symptomatic remission is defined as SFS=0 or 1 (with a 1-point reduction from baseline) and RBS=0
Endoscopic improvement is defined as MES=0 or 1
Endoscopic remission defined as a MES=0
Histologic improvement is defined as Geboes score≤3.1 [or ≤4 per Robarts Histopathology Index (RHI)]
Histologic remission is defined as Geboes score≤2A [or ≤3 per Robarts Histopathology Index (RHI)]
Histologic-endoscopic mucosal improvement (HEMI) is defined as endoscopic improvement associated with histologic improvement
Histologic-endoscopic mucosal remission (HEMR) is defined as endoscopic remission associated with histologic remission.
Conventional therapies include 5-ASA, corticosteroids (CS), immunosuppressants (IS)
Advanced therapies (AT) include biologics [anti-TNF i.e. adalimumab, infliximab, golimumab, anti-integrins i.e. vedolizumab, anti-IL23 i.e. ustekinumab], S1P receptor modulators (i.e. ozanimod), JAK inhibitors (i.e. tofacitinib, filgotinib, upadacitinib) and potential new approved drugs.
Nocturnal Bowel Movement (NBM) is defined as one or more sleep interruption due to bowel movement.
Bowel Urgency (BU) is defined as a sudden, almost uncontrollable need to go to the toilets due to bowel movements.
Extra Intestinal Manifestations (EIM) are defined as: ophthalmological manifestations (uveitis), rheumatological manifestations (axial spondyloarthritis, peripheral spondyloarthritis, arthralgia) or dermatological manifestations (psoriasis, pyoderma gangrenosum, erythema nodosum).

### Study Objectives and Endpoints

### Primary Objective and Endpoint:

The primary objective is to compare the efficacy of ABX464 versus placebo on clinical remission.

Proportion of subjects who achieve clinical remission per Modified Mayo Score at week 8.

### Key Secondary Efficacy Objectives and Endpoints

The key secondary efficacy objectives and endpoints are:
To compare the efficacy of ABX464 versus placebo on endoscopic improvement.
   Proportion of subjects who achieve endoscopic improvement at week 8.
To compare the efficacy of ABX464 versus placebo on clinical response as per MMS.
   Proportion of subjects who achieve clinical response per MMS at week 8.
To compare the efficacy of ABX464 versus placebo on symptomatic remission.
   Proportion of subjects with symptomatic remission at week 8.
To compare the efficacy of ABX464 versus placebo on histologic-endoscopic mucosal improvement (HEMI).
   Proportion of subjects with HEMI per Geboes at week 8.

Other secondary objectives and efficacy endpoints:
To compare the efficacy of ABX464 versus placebo on partial clinical response as per pMMS.
   Proportion of subjects with partial clinical response over time.
To compare the efficacy of ABX464 versus placebo on RBS.
   Proportion of subjects with no rectal bleeding over time.
To compare the efficacy of ABX464 versus placebo on SFS.
   Proportion of subjects with SFS=0 or 1 over time.
To compare the efficacy of ABX464 versus placebo on Bowel Urgency (BU).
   Proportion of subjects with BU=0 over time.
To compare the efficacy of ABX464 versus placebo on Fecal Calprotectin (FCP).
   Change from baseline in FCP over time.
   Proportion of subjects with fecal calprotectin below 150 μg/g overtime.
To compare the efficacy of ABX464 versus placebo on Fatigue Numeric Rating Scales (NRS).
   Change from baseline in the fatigue NRS over time.
To compare the efficacy of ABX464 versus placebo on FACIT-Fatigue questionnaire.
   Change from baseline in the FACIT-Fatigue at week 8.
To compare the efficacy of ABX464 versus placebo on histologic improvement.
   Proportion of subjects who achieve histologic improvement per Geboes (and RHI) scoring at week 8.
To compare the efficacy of ABX464 versus placebo on endoscopic remission.
   Proportion of subjects who achieve endoscopic remission at week 8.
To compare the efficacy of ABX464 versus placebo on histologic endoscopic mucosal remission (HEMR).
   Proportion of subjects with HEMR Geboes (and RHI) scoring at week 8.
To compare the efficacy of ABX464 versus placebo on histologic remission.
   Proportion of subjects who achieve histologic remission Geboes (and RHI) scoring at week 8.
To compare the efficacy of ABX464 versus placebo on IBDQ.
   Change from Baseline in IBDQ total score and subscores at week 8.
   Proportion of subjects with IBDQ response (increase of IBDQ≥16 from Baseline) at week 8.
To compare the efficacy of ABX464 versus placebo on nocturnal bowel movement (NBM).
   Proportion of subjects with no longer NBM at week 8 in the sub-population of subjects with NBM at baseline.
To compare the efficacy of ABX464 versus placebo on Extra Intestinal Manifestations (EIMs).
   Proportion of subjects with EIMs at week 8 in the subpopulation of subjects having EIM at baseline.
To compare the efficacy of ABX464 versus placebo on symptomatic remission.
   Proportion of subjects with symptomatic remission over time.
To compare the efficacy of ABX464 versus placebo on EQ-5D-5L.
   Change from baseline in EQ-5D-5L score over time.
To compare the efficacy of ABX464 versus placebo on hsCRP.
   Change from baseline in hsCRP over time.
To compare the efficacy of ABX464 versus placebo in WPAI.
   Change from baseline in WPAI score at week 8.
To compare the efficacy of ABX464 versus placebo on any UC related Emergency Room (ER) visits, hospitalizations and/or surgery.
   Proportion of subjects with ulcerative colitis related visits to ER, hospitalizations and/or surgery over time.
To compare the effect of ABX464 versus placebo on Total Mayo Score.
   Change from baseline in Total Mayo Score at week 8.
To compare the effect of ABX464 versus placebo on miR-124 expression in tissue and in blood.
   Change from baseline in miRNA-124 expression in rectal/sigmoidal biopsies at week 8 and in blood over time.
To compare the effect of ABX464 versus placebo on proinflammatory cytokine plasma concentrations.
   Change from baseline in proinflammatory cytokine plasma concentrations over time.
To assess the pharmacokinetic (PK) parameters of ABX464 and its main active metabolite ABX464-N-Glu after oral administration.
   Serum concentrations of ABX464 and ABX464-N-Glucuronide at baseline, week 4 and week 8 (at specified time points depending on the group allocated via IWRS).

### Safety Objective and Endpoints:

To compare ABX464 safety profile versus placebo during induction:
Incidence of all treatment-emergent adverse events (TEAEs), causally related TEAEs, all serious adverse events (SAE) and causally related SAEs classified by severity.
Incidence of adverse events leading to investigational medicinal product (IMP) discontinuation and study discontinuation.
Incidence of adverse events of special interest (AESIs).
Incidence of clinically significant laboratory abnormalities.
Incidence of clinically significant abnormalities regarding vital signs and ECG

### Cardiac Safety Sub-Study: Objectives and Endpoints

To assess the safety of ABX464 versus placebo on cardiac function assessed by echocardiography at week 8.

### Study Design and Methodology

This is a multicenter, randomized, placebo controlled study to evaluate the efficacy and safety of ABX464 given at 25 or 50 mg QD in inducing clinical remission in subjects with moderately to severely active ulcerative colitis who have inadequate response, no response, a loss of response, or an intolerance to either conventional therapies [corticosteroids, immunosuppressant (i.e. azathioprine, 6-mercaptopurine, methotrexate)] and/or advanced therapies [biologics (TNF inhibitors, anti-integrins, anti-IL-23), and/or S1P receptor modulators, and/or JAK inhibitors].

As shown below, this study consists of a screening period of up to 28 days, an 8-week induction period followed by 28-day follow up period. Subjects who complete the 8-week induction will be given the opportunity to take part in the maintenance study (i.e. ABX464-107) either:
In the randomized double-blind placebo-controlled study part [part 1; placebo, 25 mg, 50 mg] for clinical responders at week 8; and
In the open label arms study part [part 2; 25 mg or 50 mg] for non-clinical responders at week 8.

Approximately 612 subjects, aged at least 16 years, will be randomized in this study.

On Day 1, eligible subjects will be randomized and allocated into three treatment arms according to ratio 1:1:2 as follows:
Placebo: 153 subjects
ABX464—Daily dose 25 mg QD: 153 subjects
ABX464—Daily dose 50 mg QD: 306 subjects

### Study Design (See FIG. 1)

Randomization will be stratified according to the following factors:
Subjects with inadequate response to advanced therapies [AT-IR subjects](no response, loss of response or intolerance to biologics, S1P receptor modulators, JAK inhibitors) (Yes/No)
Subjects with concomitant corticosteroids at baseline (Yes/No)
Japanese subjects or non-Japanese subjects

Approximately 60% of the enrolled population should be subjects with inadequate response to Advanced Therapies (biologics, S1P receptor modulators, JAK inhibitors). In addition, percentage of subjects who have failed to respond to JAK inhibitors should be limited to 15% of subjects with inadequate response to Advanced Therapies.

Subjects with previous exposure to Advanced Therapies without a documented inadequate response will be considered as non-AT-IR subjects.

Following Day 1 (randomization day), subjects will come at the investigational site at Day 28 (Week 4) and Day 56 (Week 8) according to the study schedule of assessments.

At Day 56, subjects will be given the choice to either take part in the maintenance study (ABX464-107) or to end their participation in the present induction study by completing the study. For the latter option, subjects will return for an end of study visit 28 days after the last study drug intake. However, subjects who are eligible for the maintenance study should be encouraged to enter the maintenance study.

Subjects will be evaluated for safety and efficacy throughout the induction study.

During the screening period, a full colonoscopy will be performed for all subjects. At week 8, a flexible sigmoidoscopy is only required. At screening and week 8, during both endoscopic procedures, biopsies at the most severely affected area will be performed. Endoscopies will be centrally read. Sigmoidoscopy might be substituted by a full colonoscopy if requested by national medical practices or in case of proximal disease involvement. Biopsies will be centrally analyzed.

e-Diaries will be used to collect stool frequency, rectal bleedings, nocturnal bowel movements, bowel urgency and fatigue NRS on a daily basis, as well as the study drug intake time.

PK samples will be collected in all subjects. Subjects will be allocated into two different PK groups via IWRS. At each time point collection (D1, D28, D56) samples will be taken as follows:
for group 1: pre-dose, 0.5, 1.5, 3 h (and 6 h and/or 10 h if possible) post-dose
for group 2: pre-dose, 1, 2, 4 h (and 8 h and/or 12 h if possible) post-dose

In addition, a cardiac safety sub-study consisting in a cardiac ultrasound performed at baseline and Week 8 (and potentially over the maintenance study if the subject takes part) will be performed in selected sites with appropriate equipment and resources. Echocardiograms will be centrally read and reviewed by an Independent Cardiovascular Safety Committee. 70 subjects or more per treatment arm (coming from both induction studies) will be part of the sub-study cardiac assessment. In addition, cardiac biomarkers will be tested through induction study in all participants.

Statistical considerations and methods are specified in the SAP.

### Inclusion Criteria:

A subject will be eligible to participate in this study if ALL the following criteria are met:
1. Men or women at least 16 years old; Adolescent subjects will only be enrolled if approved by the country regulatory/health authority. If these approvals have not been granted, only subjects≥18 years old will be enrolled. To be eligible, adolescent subjects must weight≥40 kg and meet the definition of Tanner Stage 5 at the screening visit.
2. Subjects must understand, sign and date the written voluntary informed consent form at the visit prior to any protocol-specific procedures. For under-aged subjects, national requirements regarding consent should also be met.
3. Documented diagnosis of UC confirmed by endoscopy and histology. Should histology results not be available at screening, results from biopsies taken at screening may be used.
4. Active disease defined by modified Mayo score (MMS)≥5 with rectal bleeding subscore (RBS)≥1 and endoscopy subscore (MES) of 2 or 3 (confirmed by central reader).
5. Subjects with documented inadequate response (defined as lack of response or loss of response or intolerance) to at least one of the following treatments: corticosteroids, immunosuppressant, biologic therapies, S1P receptor modulators and/or JAK inhibitors and/or new drugs approved during the study (note: failure to only 5-ASA is not accepted).
   a. Inadequate response to corticosteroids (CS) is defined as either a CS resistance (i.e. signs and symptoms of persistently active disease despite current or prior course of oral prednisone/prednisolone≥40 mg/d for at least 2 weeks, or to budesonide≥9 mg/d for at least 2 weeks, or to beclomethasone≥5 mg/d for at least 2 weeks) or a CS dependence (i.e. failure to taper to ≤10 mg of prednisone/prednisolone, <9 mg/d of budesonide or <5 mg of beclomethasone or relapse occurring within 3 months after stopping CS).
      Intolerance to CS includes (but not limited to) Cushing's syndrome, osteopenia/osteoporosis, hyperglycemia, insomnia, infection.
   b. Inadequate response to immunosuppressants is defined as signs and symptoms of persistently active disease despite azathioprine treatment at 1.5 to 2.5 mg/kg/day for at least 8 weeks, or mercaptopurine 0.5 to 1.5 mg/kg/day for at least 8 weeks, or methotrexate 12.5 mg to 15 mg/week for at least 8 weeks Intolerance to immunosuppressant includes (but not limited to) nausea/vomiting, abdominal pain, pancreatitis, liver enzyme abnormalities, lymphopenia, infection.
   c. Inadequate response to biologics is defined as:
      Primary non responders to full induction course as in the USPI/SmPC of the following:
         infliximab or biosimilars (≥5 mg/kg intravenously at 0, 2, and 6 weeks),
         adalimumab or biosimilars (160 mg subcutaneous dose followed by 80 mg SC [or 80 mg SC dose in countries where this dosing regimen is allowed] followed by 40 mg SC dose at least 2 weeks apart),
         golimumab (200 mg SC dose followed by 100 mg SC dose at least 2 weeks apart),
         vedolizumab (300 mg IV at 0, 2, and 6 weeks),
         ustekinumab (one single IV using weight-based dosing −260 mg for subjects with body≤55 kg; 390 mg for subjects with body weight>55 kg and ≤85 kg; 520 mg for subjects with body weight>85 kg),
      Loss of response is defined as recurrence of symptoms during maintenance course following primary response after full induction course (discontinuation despite clinical benefit does not qualify)
      Intolerance includes (but not limited to) infusion-related reaction, serious opportunistic infection, malignancies.
   d. Inadequate response to JAK inhibitors is defined as signs and symptoms of persistently active disease despite at least one full induction course according to USPI/SmPC of tofacitinib (i.e., 8 weeks), or to at least one full induction course according to SmPC of filgotinib (i.e., 10 weeks), or to at least one full induction course according to USPI of upadacitinib (i.e., 8 weeks), or recurrence of symptoms during maintenance course.
      Intolerance to JAK inhibitor treatment that includes (but not limited to) increase of serious infection, malignancies, deep venous thrombosis (DVT), or major adverse cardiac event (MACE).
   e. Inadequate response to S1P receptor modulators is defined as signs and symptoms of persistently active disease despite at least one full induction course according to USPI/SmPC of ozanimod (i.e., at least 12 weeks) or recurrence of symptoms during maintenance course.
      Intolerance to ozanimod that includes (but not limited to) serious infections, liver enzyme, cardiac abnormalities, lymphopenia.
   f. Inadequate response or intolerance to any new drug approved for moderate to severe active UC during the course of the study defined according to USPI/SmPC.
6. Women of childbearing potential (WOCBP) subjects and male subjects with WOCBP partner must agree to use highly effective contraception methods.
7. Subjects able and willing to comply with study visits and procedures as per protocol.
8. Subjects should be affiliated to a health insurance policy whenever required by a participating country or state.

### Exclusion Criteria:

Subjects who meet ANY of the following exclusion criteria will be excluded from the study:
1. Subjects with ulcerative colitis limited to an isolated proctitis (≤15 cm from anal verge).
2. Subjects with primary sclerosing cholangitis or autoimmune hepatitis.
3. Subjects who have failed on 5-ASA therapy only.
4. Subjects with CD or presence or history of fistula, indeterminate colitis, infectious/ischemic colitis or microscopic colitis (lymphocytic and collagenous colitis).
5. History or current evidence of toxic megacolon, fulminant colitis, bowel perforation.
6. History of colon cancer, past or current evidence of low grade or high-grade colonic dysplasia and/or adenomatous polyps that have not been completely removed.
7. Recent or planned bowel surgery or history of proctocolectomy or partial colectomy or current stoma.
8. Subjects on antidiarrheals including those working on motility (e.g., loperamide, diphenoxylate with atropine, etc.).
9. Subjects on probiotics (e.g., Culturelle® [Lactobacillus GG, i-Health, Inc.], Saccharomyces boulardii).
10. Subjects who do not meet the washout period requirements prior to the screening endoscopy.
11. Subjects with the following hematological and biochemical laboratory parameters obtained during the screening period:
   Hemoglobin≤8.0 g dL⁻¹
   Absolute neutrophil count<750 mm⁻³
   Platelets<100,000 mm⁻³
   Creatinine clearance<60 mL·min⁻¹ (Cockcroft-Gault formula)
   Total serum bilirubin>1.5×ULN
   Alkaline phosphatase, aspartate aminotransferase (AST) and alanine aminotransferase (ALT)>2×ULN
12. Subjects with the following conditions (infection):
   Subjects with chronic or recurrent grade 3 or grade 4 infection within the last 2 months prior to screening or a history of opportunistic infection while not on immunosuppressive therapy.
   Herpes zoster reactivation within the last 2 months prior to screening.
   Subjects with active infection at screening or any major episode of infection that required hospitalization or treatment with intravenous antibiotics within 1 month of screening or during screening. Fungal infection of nail beds is allowed.
   Positive assay or stool culture for pathogens (ova and parasite examination, bacteria) or positive test for Clostridium difficile toxin at screening. If C. difficile is positive, subject may be treated and retested ≥2 weeks after completing treatment.
   Subject with HIV infection.
   Subjects having acute or chronic hepatitis B infection at screening (positive for hepatitis B surface antigen [HbsAg], or negative for HbsAg and positive for anti-hepatitis B core antibody in conjunction with detectable HBV DNA, or detectable HBV DNA).
   Subjects having acute or chronic hepatitis C infection at screening as defined by positive for hepatitis C antibody (subjects successfully treated and without recurrence≥1 year with no detectable HCV RNA are eligible).
   Active tuberculosis (TB) or untreated latent TB are ruled out.
13. Subjects with an uncontrolled ischemic heart disease and/or a history of congestive heart failure with New York Heart Association (NYHA) class 3 or 4 symptoms.
14. Subjects with a family or personal history of congenital or acquired long QT syndrome, or subjects with a marked baseline prolongation of QT/QTc interval (e.g., repeated demonstration of a QTc interval [Fridericia or Bazett correction]>450 milliseconds for male and >460 milliseconds for female).
15. Subjects with a history of torsade de pointe (TdP).
16. Acute or chronic of clinically relevant pulmonary, hepatic, pancreatic or renal functional abnormality, encephalopathy, neuropathy or unstable central nervous system pathology such as seizure disorder, or any other clinically significant medical problems as determined by physical examination and/or laboratory screening tests and/or medical history (note: treated autoimmune hypothyroidy and autoimmune diabetes are allowed).
17. History or active malignancy (subjects with a 5-year disease free survival are eligible).
18. Serious illness requiring hospitalization within 4 weeks prior to screening (except UC flare).
19. Subjects previously treated with ABX464.
20. WOCBP subject and WOCBP partner of male subject who are pregnant at screening, intend or are planning to become pregnant during the study duration, and breast-feeding women.
21. Illicit drug or alcohol abuse or dependence.
22. Subjects who received live vaccine within 3 months prior to screening and/or who's planning to receive such a vaccine during the study duration.
23. Use of any investigational or non-registered product within 3 months or within 5 half-lives preceding baseline, whichever is longer, and during the study.
24. Any condition, which in the opinion of the investigator, could compromise the subject's safety or adherence to the study protocol.

### IMP, Dose, and Mode of Administration

Depending on the treatment arm allocation, ABX464/Placebo (capsules) 50 mg and 25 mg are administered once daily in the morning with food for 8 weeks during the induction phase.

### Medications

All previous UC medications on which failure of treatment has occurred will be recorded into the eCRF without time limitation for advanced therapies and over the past 2 years for conventional treatments. Previous and current medications will be reviewed to check the use of potentially prohibited medications and ensure appropriate washout periods are respected. All concomitant medications will be recorded into the eCRF.

### Allowed Concomitant UC Medications

For the treatment of UC (provided stable dose for at least 2 weeks prior to the screening endoscopy):
Corticosteroids: prednisone or prednisone equivalent≤15 mg/day; beclomethasone dipropionate (≤5 mg/day) or budesonide MMX (≤9 mg/day).
Oral 5-ASA.

### Prohibited Previous and Concomitant Medications

The following drugs are prohibited from enrollment, through the study, until study completion. For some of them used prior to subjects' enrollment a strict washout period may apply. Sites are allowed to assay drug levels during the study period to shorten the washout periods.

Ustekinumab must be stopped at least 12 weeks prior to Screening endoscopy.

Infliximab, adalimumab, golimumab, vedolizumab and ozanimod must be stopped at least 8 weeks prior to Screening endoscopy.

Cyclosporine, tacrolimus, mycophenolate mofetil, or thalidomide must be stopped at least 4 weeks prior to Screening endoscopy.

Parenteral or enteral nutrition must be stopped at least 3 weeks prior to screening endoscopy.

Tofacitinib, filgotinib, upadacitinib must be stopped at least 2 weeks prior to Screening endoscopy.

Rectal aminosalicylates or corticosteroids, other enemas/suppositories (other than required for endoscopy), must be stopped at least 2 weeks prior to the Screening endoscopy.

Azathioprine or 6-mercaptopurine or methotrexate must be stopped at least 2 weeks prior to Screening endoscopy.

Intravenous corticosteroids must be stopped at least 2 weeks prior to Screening endoscopy.

UC-related antibiotics that have been discontinued must be stopped at least 2 weeks of Screening endoscopy.

Topical (i.e. rectal) corticosteroids and/or 5-ASA must be stopped at least 2 weeks prior to Screening endoscopy

Antidiarrheals including those working on motility (e.g., loperamide, diphenoxylate with atropine) must be stopped at least 2 weeks prior to Screening endoscopy. Any use of antidiarrheals for acute and severe diarrhea must be documented in the dedicated eCRF.

Probiotics, fish oil, fecal transplantation must be stopped at least 2 weeks prior to Screening endoscopy.

Traditional Chinese medicine must be stopped at least 2 weeks prior to Screening endoscopy.

Non-steroidal anti-inflammatory drugs (NSAIDs) must be stopped at least 1 week prior to Screening endoscopy and during the study (except topical NSAIDs and the use of low dose aspirin for cardiovascular [CV] protection or short course [less than 7 days] of NSAIDs for treatment of adverse event).

Vaccination with live components is prohibited during the course of the study and up to 8 weeks after the last ABX464 dosing (of note, COVID vaccination is allowed with the exception of live-attenuated vaccine).

Drugs that inhibit or induce CYP1A2.

Drugs that inhibit UDP-glucuronosyltransferase (UGT) IA9 activity and inhibitors of organic anion transporter (OAT) P1B1/P1B3 transporters.

Natalizumab: no previous exposure allowed.

Use of any investigational or non-registered product is prohibited within 3 months or within 5 half-lives preceding baseline, whichever is longer and during the study.

### Premature Trial Discontinuation

Subject's premature trial discontinuation must occur for the following reasons:
An AE or an intercurrent condition that precludes continuation of treatment.
Specifically, an increase≥3.0×ULN in liver transaminases (AST and/or ALT) or an increase≥2.0×ULN in alkaline phosphatase or in total bilirubin requires close observation with repeating liver enzymes and serum bilirubin tests twice weekly and clinical investigation to understand the etiology of this elevation. Frequency of retesting can decrease to once a month if abnormality stabilizes after the initial 2 weeks of follow-up and if the subject is asymptomatic. Discontinuation of the study treatment should occur if:
   ALT or AST>8×ULN.
   ALT or AST>5×ULN for more than 2 weeks.
   ALT or AST>3×ULN and total bilirubin>2×ULN or INR>1.5.
   ALT or AST>3×ULN with appearance of fatigue, nausea, vomiting, right upper quadrant pain or tenderness, fever, rash, and/or eosinophilia (>5%).
Severe (grade 3 or higher) infection, a severe (grade 3 or higher) opportunistic infection, or sepsis.
Any medically significant abnormal laboratory results, including new onset anemia (defined as a hemoglobin decrease>2 g/dL from baseline or hemoglobin<8 g/dL)—note: transient abnormal values, and/or abnormal values related to an existing condition that doesn't constitute a medically significant worsening will not be a criterion for discontinuation.
Onset of acute pancreatitis (as defined by Atlanta criteria).
Any cardiac AESI or any cardiac condition diagnosed during the course of the study and assessed by the Independent Cardiovascular Safety Committee as changing the risk/benefit balance for the subject.
Any malignancies, including dysplastic skin lesions.
Any relevant toxicity or negative change in the risk/benefit assessment leading to an unacceptable risk for the subject (i.e., occurrence of AEs which character, severity or frequency is new in comparison to the existing risk profile), or any data deriving from other clinical trials or toxicological studies which negatively influence the risk/benefit assessment.
Pregnancy.
Investigator's decision.
Withdrawal of consent.
Administrative reasons from Sponsor (study level).

In case of premature discontinuation, subjects will be invited to perform an End Of Treatment (EOT) visit as per study scheduled of assessment.

### Subject Follow-Up

For completed subjects, the last dose of the induction study will be taken the day before the last visit (D56±2 days).

Subjects eligible for maintenance will be encouraged to take part in the ABX464-107 maintenance. The first treatment dose taken in the frame of the maintenance study will be taken after the study specific informed consent is signed.

Subjects not willing to take part in the maintenance study will come for a last safety follow-up visit and will be then followed as per the site standard of care after this last study visit.

### Sample Size Calculation

A total of 612 subjects (153:153:306) will allow the detection of a difference of at least 13% in the clinical remission rate at W8 between ABX464 50 mg (and 25 mg) and placebo with a statistical power of at least 90%. This calculation, based on a Chi-square test with a type I error rate of 5% (two-sided), assumes a placebo clinical remission rate of 8%. The reason for having the 1:1:2 allocation is to generate a sufficient number of subjects on ABX464 50 mg with clinical response and to be enrolled into the maintenance study.

Sample size calculations was performed with nQuery, version 7.

### Statistical Methods

### Efficacy:

The primary endpoint, clinical remission (based on the modified Mayo Scoring system, is defined as SFS=0 or 1 and no greater than baseline and RBS=0 and endoscopy subscore=0 or 1 (friability is scored as 2), will be analyzed using a Cochran Mantel Haenszel test (CMH) test adjusting for AT-IR (yes/no); baseline corticosteroid treatment and Japanese or non-subjects as stratification factors, using the ITT population.

Comparisons on the primary endpoint will be analyzed in sequence (clinical remission rate at Week 8 between the 50 mg dose group vs placebo and then clinical remission rate at Week 8 between the 25 mg dose group vs placebo).

If comparisons on primary endpoint for each dose are statistically significant (0.05 level, 2-sided situation), then, this testing procedure continued through each of the key secondary endpoints in the prespecified order until an endpoint failed to reach statistical significance (0.05 level, 2-sided situation), after which all subsequent key secondary endpoints would be considered exploratory.

Intercurrent events considered for primary estimand and their corresponding management are: premature discontinuation of study drug (composite policy), prohibited change in UC medications (composite variable strategy-NRI), premature discontinuation from study (composite variable strategy-NRI).

Sensitivity analyses, addressing estimand strategies together with the corresponding estimands will be performed.

### Safety:

AEs will be tabulated (counts and percentages) by group. All AEs will be listed, and the data will be tabulated by body system/organ class. AE tabulations will include all TEAEs, which will be further classified by severity and relationship to treatment and dose level.

Clinical laboratory parameters, vital signs, ECG will be summarized by using descriptive statistics (n, mean, SD, SEM, median, minimum and maximum). The number of subjects with at least one abnormal value will be tabulated (counts and percentages) for each parameter in summary shift tables, by group and dose.

### Pharmacokinetics:

A popPK model will be constructed for analysis of PK data.

### End of Trial Definition

If the last subject takes part to the maintenance study: End of Trial will be based on the last visit of the last remaining subject in the induction study.

If the last subject will not take part in the maintenance study, End of Trial will be the date of the last follow-up of the last remaining participant. In case of last subject is declared lost to follow-up, the date of the last follow-up or the date of the last contact attempt (whichever occurs later) should be considered.

### Example 3. A Randomized, Double-Blind, Multicenter Phase III Study to Evaluate the Long-Term Efficacy and Safety of ABX464 25 mg or 50 mg Once Daily as a Maintenance Therapy in Subjects with Moderately to Severely Active Ulcerative Colitis (ABX464-107)

### Study Specific Definitions

For the purpose of the study, the following definitions will be applied:
Modified Mayo Score (MMS) is defined as 3-component Mayo Score: Rectal bleeding sub-score (RBS), Stool Frequency sub-score (SFS) and Mayo Endoscopic Sub-score (MES).
Partial Modified Mayo Score (pMMS) is defined as combination of RBS and SFS.
Clinical remission per MMS is defined as SFS=0 or 1 and RBS=0 and MES=0 or 1.
Sustained clinical remission is defined as clinical remission assessed at Week 44 in the sub population of subjects in clinical remission at maintenance study entry.
Corticosteroid-free clinical remission is defined as clinical remission at Week 44 and corticosteroid free for at least 12 weeks prior to Week 44 in the subpopulation with corticosteroids at maintenance study entry
Clinical response per MMS is defined as a reduction from baseline in MMS≥2 points and ≥30% associated to a reduction≥1 point in RBS and/or RBS=0 or 1.
Sustained clinical response is defined as clinical response assessed at Week 44 in the sub population of subjects in clinical response at maintenance study entry.
Partial Clinical response per pMMS is defined as a reduction from baseline in pMMS≥1 points and ≥30% associated to a reduction≥1 point in RBS and/or RBS=0 or 1.
Symptomatic remission is defined as SFS=0 or 1 and RBS=0.
Endoscopic improvement is defined as Mayo Endoscopic Subscore (MES)=0 or 1.
Sustained endoscopic improvement is defined as endoscopic improvement assessed at Week 44 in the sub population of subjects with endoscopic improvement at maintenance study entry. Evaluation will be based on the worst affected segment.
Endoscopic remission defined as a MES=0.
Histologic improvement is defined as Geboes score≤3.1 [or ≤4 per Robarts Histopathology Index (RHI)].
Histologic remission is defined as Geboes score≤2A [or ≤3 per Robarts Histopathology Index (RHI)].
Histologic-endoscopic mucosal improvement (HEMI) is defined as endoscopic improvement associated with histologic improvement.
Histologic-endoscopic mucosal remission (HEMR) is defined as endoscopic remission associated with histologic remission.
Conventional therapies include 5-ASA, corticosteroids (CS), immunosuppressants (IS)
Advanced therapies include biologics (anti-TNF i.e., adalimumab, infliximab, golimumab, anti-integrins i.e., vedolizumab, anti-IL23 i.e., ustekinumab) and S1P modulators and JAK inhibitors.
Relapse is defined as an increase≥2 points in partial MMS (pMMS) from the maintenance baseline at 2 separate occasions at least 2 weeks apart with a 1-point increase of the RBS compared to maintenance baseline.
Disease worsening is defined as an increase≥5 points in MMS with MES=2 to 3.
Maintenance baseline: All the assessments performed at week 8 of the induction studies will be considered as baseline assessments for Maintenance.
Nocturnal bowel movement (NBM) is defined as one or more sleep interruption due to bowel movement.
Bowel urgency (BU) is defined as a sudden, almost uncontrollable need to go to the toilets due to bowel movements.
Extra Intestinal Manifestations (EIM) are defined as: ophthalmological manifestations (uveitis), rheumatological manifestations (axial spondylarthritis, peripheral spondylarthritis, arthralgia) or dermatological manifestations (Psoriasis, pyoderma gangrenosum, Erythema nodosum).

### Study Objectives and Endpoints

The Maintenance study is divided into 2 parts:
Part #1—double-blind placebo controlled: for subjects in clinical response at the end of the induction study (i.e., ABX464-105 or ABX464-106)
Part #2—double-blind uncontrolled: for clinical non-responder subjects at the end of the induction study (i.e., ABX464-105 or ABX464-106). Also, subjects from Part #1 who are in relapse will enter Part #2 once relapse is confirmed.

Primary and key secondary endpoints will be adequately adjusted for multiplicity to protect the type-I error rate using a mixture gatekeeping procedure. Details are provided in the statistical analysis plan. Primary and key secondary efficacy objectives & endpoints detailed below are applicable to Part #1 of the maintenance study. Similar endpoints will be used for Part #2 but will be presented in a descriptive manner.

### Primary Objective and Endpoint:

To evaluate the efficacy of ABX464 versus placebo on clinical remission.

Proportion of subjects in clinical remission at Week 44.

### Key Secondary Efficacy Objectives and Endpoints

To evaluate the efficacy of ABX464 versus placebo on endoscopic improvement.
   Proportion of subjects with endoscopic improvement at Week 44
To evaluate the efficacy of ABX464 versus placebo on symptomatic remission.
   Proportion of subjects with symptomatic remission at Week 44
To evaluate the efficacy of ABX464 versus placebo on corticosteroid-free clinical remission.
   Proportion of subjects with corticosteroid-free clinical remission at Week 44.
To evaluate the efficacy of ABX464 versus placebo to sustain clinical remission.
   Proportion of subjects with sustained clinical remission at Week 44.
To evaluate the efficacy of ABX464 on histologic-endoscopic mucosal improvement (HEMI) versus placebo.
   Proportion of subjects with HEMI per Geboes scoring at Week 44
To evaluate the efficacy of ABX464 versus placebo on endoscopic remission.
   Proportion of subjects with endoscopic remission at Week 44.

### Other Secondary Objectives and Efficacy Endpoints:

To evaluate the efficacy of ABX464 versus placebo on histologic improvement versus placebo.
   Proportion of subjects with histologic improvement per Geboes and RHI scoring at Week 44.
To evaluate the efficacy of ABX464 versus placebo on histologic remission versus placebo.
   Proportion of subjects with histologic remission per Geboes and RHI scoring at Week 44.
To evaluate the efficacy of the different dose groups of ABX464 on histologic-endoscopic mucosal remission (HEMR) versus placebo.
   Proportion of subjects with HEMR per Geboes and RHI scoring at Week 44
To compare the efficacy of ABX464 versus placebo on fecal calprotectin (FC) level.
Change from baseline in fc levels over time
   Proportion of subjects with FC below 150 μg/g over time
To compare the efficacy of ABX464 versus placebo on IBDQ
   Change from Baseline in IBDQ total and domain score over time.
   Change from Baseline in individual IBDQ item under Bowel Symptom domain (for Q1, Q5, Q9, Q13, Q17, Q20, Q22, Q24, Q26, and Q29) over time.
   Proportion of subjects with IBDQ response (increase of IBDQ≥16 from Baseline) over time.
      Proportion of Subjects with IBDQ Remission (IBDQ Total Score≥170) Over Time.
To compare the efficacy of ABX464 versus placebo on nocturnal bowel movement (NBM)
   Proportion of subjects with no more NBM at Week 44 in the sub-population of subjects with NBM at Week 8.
To evaluate the efficacy of ABX464 versus placebo on Extra Intestinal Manifestations (EIM)
   Proportion of subjects with EIM at Week 44 in the sub-population of subjects with EIM at Week 8.
To evaluate the efficacy of ABX464 versus placebo on partial Clinical response
   Proportion of subjects in partial clinical response over time
To evaluate the efficacy of ABX464 versus placebo on symptomatic remission.
   Proportion of subjects with symptomatic remission over time
To evaluate the efficacy of ABX464 on rectal bleeding.
   Proportion of subjects with an RBS=0 over time
To evaluate the efficacy of ABX464 on stool frequency.
   Proportion of subjects with an SFS=0 or 1 over time
To evaluate the efficacy ABX464 on bowel urgency.
   Proportion of subjects with no bowel urgency over time.
To evaluate the efficacy of ABX464 versus placebo on clinical response as per MMS.
   Proportion of subjects who achieve clinical response per MMS at Week 44.
To evaluate the efficacy of ABX464 versus placebo to sustain clinical response as per MMS.
   Proportion of subjects who achieve sustained clinical response per MMS at Week 44.
To compare the efficacy of ABX464 versus placebo in ulcerative colitis related visits to the Emergency Room (ER) hospitalizations and/or surgery.
   Proportion of subjects with ulcerative colitis related visits to ER, hospitalizations and/or surgery during maintenance.
To evaluate the efficacy of ABX464 versus placebo on the fatigue NRS
   Change from baseline in the fatigue NRS score over time.
To evaluate the efficacy of ABX464 versus placebo on the FACIT-fatigue
   Change from baseline in the FACIT-fatigue score at Week 44.
To evaluate the efficacy of ABX464 versus placebo on high sensitive Creactive protein (hs-CRP) levels.
   Change from maintenance baseline in hs-CRP levels at Week 44
To evaluate the efficacy of ABX464 versus placebo on Quality of Life (QoL) measured EuroQoL-5 Dimensions questionnaire (EQ-5D-5L).
   Change from maintenance baseline in EQ-5D-5L questionnaire at Week 28 and Week 44.
To evaluate the efficacy of ABX464 versus placebo on subjects work productivity and activity measured the Work Productivity and Activity Impairment questionnaire (WPAI).
   Change from maintenance baseline in WPAI questionnaire at Week 28 and Week 44.
To evaluate the efficacy of ABX464 versus placebo on miR-124 expression in tissue (RNA later).
   Change from maintenance baseline in miRNA-124 expression in rectal/sigmoidal biopsies at Week 44
      Change from Baseline in Total Blood Over Time.
To evaluate the efficacy of the different dose groups of ABX464 versus placebo on proinflammatory cytokines plasma concentrations.
   Change from maintenance baseline in pro-inflammatory cytokines plasma concentrations over time.

### Cardiac Safety Sub-Study: Objectives and Endpoints

The cardiac safety sub-study objective is to assess the safety of ABX464 versus placebo on cardiac function evaluated by echocardiography at week 28 and week 44.

### Safety Objectives and Endpoints (Part #1 and #2):

To evaluate the safety profile of the different dose groups of ABX464 versus placebo:
Incidence of all treatment-emergent adverse events (TEAEs), causally related adverse events (AEs), all serious adverse events (SAEs) and causally related SAEs classified, by severity
Incidence of AEs leading to investigational medicinal product discontinuation and study discontinuation.
Incidence of adverse events of special interest (AESIs).
Incidence of clinically significant laboratory abnormalities.
Incidence of clinically significant abnormalities regarding vital signs and ECG.

### Study Design and Methodology

This is a multicenter, randomized study to evaluate the long-term efficacy and safety of ABX464 50 mg and 25 mg administered once daily (QD) as maintenance therapy in subjects with moderately to severely active ulcerative colitis who have inadequate response, no response, a loss of response, or an intolerance to either conventional therapies [corticosteroids, immunosuppressant (i.e. azathioprine, 6-mercaptopurine, methotrexate)] and/or advanced therapies [biologics (TNF inhibitors, anti-integrins, anti-IL-23), and/or S1P receptor modulators, and/or JAK inhibitors].

This study is the maintenance phase of both previous induction studies ABX464-105 and ABX464-106.

All eligible subjects who have completed either induction study (ABX464-105 or ABX464-106), will be encouraged to participate to the present ABX464-107 maintenance study. All subjects will be either randomly allocated as induction clinical responders to a double blind, placebo-controlled substudy (Part #1) or allocated as induction clinical non-responders to the double blind uncontrolled sub-study (Part #2). This maintenance study (Parts #1 and #2) consists of a 44-week treatment phase and a 28-day follow-up period concluding with the End of Study (EOS) visit.

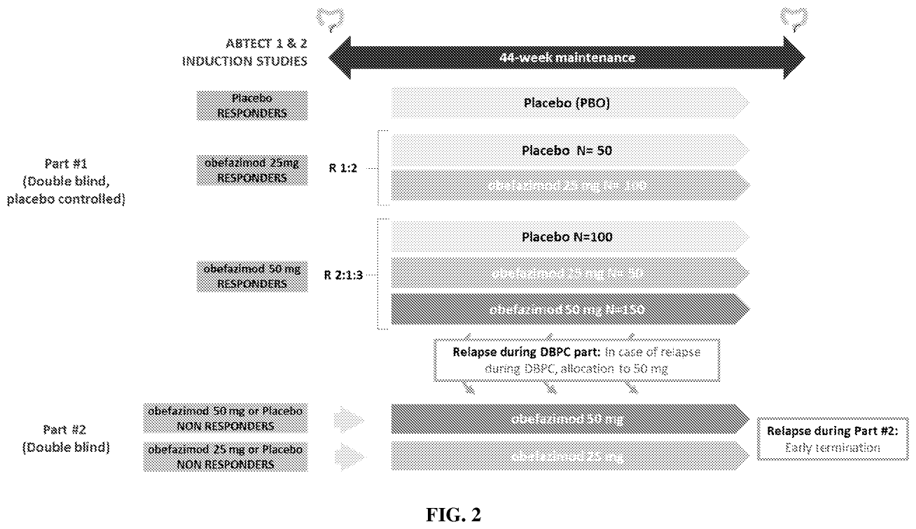

### Study Design (See FIG. 2)

Subjects' eligibility will be confirmed at the end of the induction study (i.e., either ABX464-105 or ABX464-106). Entry criteria will mainly be based on subject's willingness to participate to the maintenance study and induction study completion.

Subjects experiencing a clinical response (i.e., responders) at the end of the induction studies will be randomized into study Part #1, while non-responder subjects will be randomized/allocated into study Part #2.

Among both induction studies, approximatively 450 subjects (excluding placebo responder subjects) are expected to be assessed as responders and randomized as follows according to their induction treatment:
ABX464—Dose 50 mg QD: approximatively 150 subjects
ABX464—Dose 25 mg QD: approximatively 150 subjects (100 subjects from the 25 mg induction dose group and 50 subjects from the 50 mg induction group)
Placebo: approximatively 150 subjects (50 subjects from the 25 mg induction dose group and 100 subjects from the 50 mg induction group)

Subjects with clinical response on active treatment in the induction studies will have the 2:1 odd, i.e., 2/3 chance of being randomized to active treatment during maintenance. Subjects with clinical response and who were dosed with placebo in the induction studies will continue to receive placebo during the maintenance treatment (blinded).

Randomization will be stratified according to the following:
Clinical Remission status (yes/no)
Induction dose (50 mg or 25 mg)
Subject who are treated with oral corticosteroids at baseline of the maintenance (yes/no)

Subjects without a clinical response at the end of the induction studies (600 subjects expected) may opt in the maintenance study Part #2. They will be randomized/allocated into the two ABX464 blinded dose levels: ABX464—Dose 50 mg QD or ABX464—Dose 25 mg QD

Allocation/randomization will be performed according to the following:
Non-responder subjects who had received 50 mg during induction will be allocated into the 50 mg blinded dose group
Non-responder subjects who had received 25 mg during induction will be allocated into the 25 mg blinded dose group
Non-responder subjects who had received placebo during induction will be randomized into the 25 mg and 50 mg blinded dose groups according to a 1:1 ratio

From Day 1 onwards, subjects will be seen at the investigational site at Week 4, (and at Week 8 for Part #2 subjects only), then every 8 weeks up to Week 44 and 4 weeks after the last dose for an end of study visit. An end of study visit will also be performed should the subjects not be eligible for the long-term extension safety study.

For subjects joining Part #2 after relapse in Part #1, if there is no scheduled visit within 4 weeks (+/−2 weeks), a site visit should be performed 4 weeks later as an unscheduled visit (week 4, SoA, Table 5.1).

At week 44, a flexible sigmoidoscopy with biopsies from the most severely affected area will be performed. If lesions are healed, biopsies should be taken from approximately the same area of the baseline endoscopy. Endoscopies will be centrally read. Sigmoidoscopy might be substituted by a full colonoscopy if requested by national medical practices and/or guidelines. Biopsies will be also centrally analyzed.

e-Diaries will be used to collect stool frequency, rectal bleedings, nocturnal bowel movements, bowel urgency, fatigue NRS as well as study drug intake times.

Should a subject experience an UC relapse and depending on the study part, the following rules must be applied:
For Part #1: at a given visit, should a subject meet the relapse definition (MMS increase≥2 points from Maintenance baseline with an increase of the rectal bleeding score increase≥1 point) the subject will be invited to come back to the site approximately 2 weeks later to perform a “relapse confirmatory visit” (RCV).
If the relapse is not confirmed, the subject will remain in Maintenance part #1.
If the relapse is confirmed, the subject must:
   Exit the Maintenance Part #1 and be allocated into Maintenance Part #2 into the ABX464 50 mg QD dose group.
   Participate to an endoscopy within approximately 6 weeks after the RCV to check whether the subject meets the disease worsening definition (local read). It is allowed to perform the endoscopy the same day as RCV if medically justified as per investigator's judgement. In any case, endoscopy should not be done more than 8 weeks after RCV.
   If not met, the subject will remain in Maintenance Part #2 and be treated with ABX464 50 mg QD.
   If the disease worsening definition is met, the subject will be invited to perform an End of Treatment (EoT) visit and will come back 4 weeks later for an End of Study (EoS) visit.
If a subject meets the relapse definition during Maintenance Part #2 (blinded ABX464 treatment groups) a confirmatory visit should be done 2 weeks later. If the relapse is confirmed, then an unscheduled endoscopy should be performed within 2 to 4 weeks after RCV. Should the subject meet the definition of disease worsening s/he must be withdrawn from the study and stop the study treatment. Part #2 subjects will be invited to perform all the End of Treatment (EoT) visit assessments (except cytokines and miRNA) and will return 4 weeks later for an End of Study visit (EOS).

### Maintenance Cardiac Sub-Study:

Subjects who had an echocardiography during the induction study will undergo additional echocardiographies at Week 28 and at Week 44. During the course of the maintenance study, should a subject from this sub-group discontinue treatment early, an echocardiography will be performed at the EOS visit.

### Inclusion Criteria:

A subject will be eligible to participate in this study if ALL the following criteria are met:
1. Subjects must have completed the induction treatment study (ABX464-105 or ABX464 106), and subjects' clinical response status must be available.
2. Subjects with a valid endoscopy performed at the end of the induction study and results from central reader available at Day 1.
3. Subjects must understand, sign and date the written voluntary informed consent form at the visit prior to any protocol-specific procedures. For under-aged subjects, national requirements regarding consent should also be met.
4. Women of childbearing potential (WOCBP) subjects and male subjects with WOCBP partner must agree to use highly effective contraception methods.
5. Subjects must be able and willing to comply with study visits and procedures as per protocol.
6. Subjects should be affiliated to a health insurance policy whenever required by a participating country or state.

### Exclusion Criteria:

Subjects who meet ANY of the following exclusion criteria will be excluded from the study:
1. Subjects who permanently discontinued the study treatment during the induction study (either ABX464-105 or ABX464-106).
2. Subjects who have developed any major illness/condition or evidence of an unstable clinical condition (except UC) during the induction study that, in the investigator's judgment, will substantially increase the risk to the participant if he or she participates in the study.
3. Subjects who plan to participate in other investigational studies during the maintenance study.
4. Pregnant or breast-feeding women or male subject of a pregnant partner,
5. Male or female subjects planning a pregnancy within the coming 12 months.
6. Subjects who have not adhered to abstinence of prohibited and/or concomitant medications.
7. Any changes in the laboratory values during the induction period that correspond with the following:
   Hemoglobin≤8.0 g dL⁻¹
   Absolute neutrophil count<750 mm-3
   Platelets<100,000 mm-3
   Creatinine clearance<60 mL·min-1 (Cockcroft-Gault formula)
   Total serum bilirubin>1.5×ULN
   Alkaline phosphatase, aspartate aminotransferase (AST) and alanine aminotransferase (ALT)>2×ULN

### Test Product, Dose, and Mode of Administration

Depending on the treatment arm allocation, ABX464 dose 50 mg or 25 mg (or its matching placebo) will be administrated QD in fed condition (ideally at the same time in the morning) for 44 weeks during the maintenance phase.

### Medications

All previous UC medications on which failure of treatment has occurred will be recorded into the eCRF without time limitation for advanced therapies and over the past 2 years for conventional treatments.

### Allowed UC Concomitant Medications

For the treatment of UC (provided stable dose):
Corticosteroids: prednisone or prednisone equivalent≤15 mg/day; beclomethasone dipropionate (≤5 mg/day) or budesonide MMX (≤9 mg/day).
Oral 5-ASA.

### Tapering of Corticosteroids

Part #1 subjects who enter the maintenance study on corticosteroid therapy for treatment of their UC and who achieved clinical response in the induction studies must initiate corticosteroid tapering, as described below, from Day 1 onwards of the maintenance study.

Part #2 clinical non-responders who start blinded ABX464 treatment will begin corticosteroid tapering if symptomatic improvement, based on investigator discretion, is evident at any time after starting the blinded ABX464 treatment.

For subjects who cannot tolerate the corticosteroid taper because of clinical symptoms, corticosteroid taper may be paused and/or corticosteroid dose may be increased up to the original dose at induction baseline (should not exceed induction baseline dose). In such case, attempts to reinitiate corticosteroid tapering should be made within 2 weeks of interruption of taper.

The recommended tapering schedule for oral corticosteroids (other than budesonide extended-release tablets [budesonide MMX]) is as follows:
Dose>10 mg/day prednisone or equivalent: taper daily dose by 5 mg/week until receiving 10 mg/day, and then continue tapering at 2.5 mg/week until 0 mg/day.
Dose≤10 mg/day prednisone or equivalent: taper daily dose by 2.5 mg/week until 0 mg/day.

The recommended tapering schedule for subjects receiving oral budesonide MMX 9 mg/day is to reduce tablets to 9 mg every other day for 2 weeks, followed by 9 mg every third day for 2 weeks, and then discontinue.

### Prohibited Previous and Concomitant Medications

Any change in concomitant medication will be duly checked at each study visit to avoid any protocol deviation. At enrollment, no washout period is applicable considering this study is the maintenance study following induction periods via ABX464-105 and ABX464-106 induction studies where the following medications are strictly prohibited during the course of both trials.
Rectal amino salicylates or corticosteroids, other enemas/suppositories (other than required for endoscopy).
Cyclosporine, tacrolimus, mycophenolate mofetil, or thalidomide.
Azathioprine, 6-mercaptopurine or methotrexate.
UC-related antibiotics.
Infliximab, ozanimod, adalimumab, and golimumab.
Vedolizumab and ustekinumab.
Non-steroidal anti-inflammatory drugs (NSAIDs) during the study (except topical NSAIDs and the use of low dose aspirin for cardiovascular [CV] protection; except short-term treatment course [less than 7 days] for Adverse Event treatment).
Traditional Chinese medicine.
Tofacitinib, filgotinib, upadacitinib.
Antidiarrheals (e.g., loperamide, diphenoxylate with atropine). Any use of antidiarrheals for acute and severe diarrhea must be documented in the dedicated eCRF.
Probiotics, fish oil, fecal transplantation.
Vaccination with live components is prohibited during the course of the study and up to 8 weeks after the last ABX464 dosing (of note, COVID vaccination is allowed with the exception of live-attenuated vaccine).
Natalizumab.
Drugs that inhibit or induce CYP1A2.
Drugs that inhibit UDP-glucuronosyltransferase (UGT)IA9 activity and inhibitors of organic anion transporter (OAT)P1B1/1B3 transporters.
Use of any investigational or non-registered product during the study.

### Premature Trial Discontinuation

Subject's premature trial discontinuation must occur for the following reasons:
An AE or an intercurrent condition that preclude continuation of treatment.
Specifically, an increase≥3.0×ULN in liver transaminases (AST and/or ALT) or an increase≥2.0×ULN in alkaline phosphatase or in total bilirubin requires close observation with repeating liver enzymes and serum bilirubin tests twice weekly and clinical investigation to understand the etiology of this elevation. Frequency of retesting can decrease to once a month if abnormality stabilizes after the initial 2 weeks of follow-up and if the subject is asymptomatic. Discontinuation of the study treatment should occur if:
   ALT or AST>8×ULN.
   ALT or AST>5×ULN for more than 2 weeks.
   ALT or AST>3×ULN and total bilirubin>2×ULN or INR>1.5.
   ALT or AST>3×ULN with appearance of fatigue, nausea, vomiting, right upper quadrant pain or tenderness, fever, rash, and/or eosinophilia (>5%).
Severe (grade 3 or higher) infection, a severe (grade 3 or higher) opportunistic infection, or sepsis.
Any medically significant abnormal laboratory results, including new onset anemia (defined as a hemoglobin decrease≥2 g/dL from baseline or hemoglobin<8 g/dL)—note: transient abnormal values, and/or abnormal values related to an existing condition that doesn't constitute a medically significant worsening will not be a criterion for discontinuation.
Onset of acute pancreatitis (as defined by Atlanta criteria).
Any cardiac AESI or any cardiac condition diagnosed during the course of the study and assessed by the Independent Cardiovascular Safety Committee as changing the risk/benefit balance for the subject.
Any malignancies, including dysplastic skin lesions.
Any relevant toxicity or negative change in the risk/benefit assessment leading to an unacceptable risk for the subject (i.e., occurrence of AEs which are characterized by increased severity or frequency and are new in comparison to the existing risk profile), or any new data from other clinical trials or toxicological studies which negatively influence the risk/benefit assessment.
Pregnancy.
Disease worsening.
Investigator's decision.
Withdrawal of consent.
Administrative reasons from Sponsor (study level).

In case of premature discontinuation, subjects will be invited to perform an End of Treatment visit as per study schedule of assessment.

### Subject Follow-Up

For completed subjects, the last dose of the maintenance study will be taken the day of week 44 visit. A safety follow-up visit will take place 4 weeks after the last study drug intake. A separate long-term extension safety study, will be proposed to treatment responder subjects at the end of treatment visit, should all the regulatory authorizations be obtained.

All subjects will be followed as per the site standard of care.

### Sample Size Calculation

Taking into consideration the impact of the strategy to reflect intercurrent events, a total of around 450 subjects (150:150:150) will allow the detection of a difference of at least 20% in the clinical remission rate at W44 between each dose of ABX464 and placebo, assuming a clinical remission rate of 25% for the placebo, with a statistical marginal power of at least 90%. This calculation is based on a Chi-square test with a type I error rate of 2.5% two-sided to ensure that at least one of the 2 doses will pass the first family.

Sample size calculations was performed with nQuery, version 7.

### Statistical Methods

### Efficacy:

The primary endpoint, clinical remission [SFS=0 or 1, RBS=0 and endoscopy sub-score=0 or 1 (friability is scored as 2)] at Week 44, will be analyzed using a Cochran Mantel Haenszel test (CMH) test adjusting for clinical remission (yes/no), baseline corticosteroid (yes/no), induction treatment (ABX464 25 mg or 50 mg), using the ITT analyses set.

Estimands will be detailed in the SAP.

The primary endpoint will be tested using (controlled) an overall type I error rate of 0.05, and a two-sided test. Results will be presented using the frequency and the proportion by treatment group with the difference between proportions adjusted for stratification factors, and the OR together with its corresponding 95% confidence interval. Data will be presented for non-responder imputation, NRI, in summary presentations of analyses.

Sensitivity analyses, addressing estimands strategies together with the corresponding estimands will be done, and presented in the SAP.

### Safety:

AEs will be tabulated (counts and percentages) by group. All AEs will be listed, and the data will be tabulated by body system/organ class. AE tabulations will include all TEAEs, which will be further classified by severity and relationship to treatment and dose level.

Clinical laboratory parameters, vital signs, ECG will be summarized by using descriptive statistics (n, mean, SD, SEM, median, minimum and maximum). The number of subjects with at least one abnormal value will be tabulated (counts and percentages) for each parameter in summary shift tables, by group and dose.

### End of Trial Definition

End of Trial will be the date of the last follow-up of the last remaining participant. In case of last subject is declared lost to follow-up, the date of the last follow-up or the date of the last contact attempt (whichever occurs later) should be considered.

### Example 4. A Follow-Up Phase II Open-Label Study to Evaluate the Long-Term Safety and Efficacy Profile of ABX464 Given at 25 mg Once Daily in Subjects with Moderate to Severe Active Ulcerative Colitis (ABX464-108)

### Study Objectives and Endpoints

### Primary Objective and Endpoint:

The primary objective of the study is to evaluate the long-term safety of ABX464 given at 25 mg once daily in subjects with Moderate to Severe Active Ulcerative Colitis
   Number of Adverse Events (AEs) in ABX464 treated subjects

### Secondary Objectives and Endpoints

The secondary objectives are:
To evaluate the long-term effect of ABX464 on clinical remission;
   Percentage of subjects reaching clinical remission at yearly visits.
To evaluate the long-term effect of ABX464 on endoscopic remission;
   Percentage of subjects reaching endoscopic remission at yearly visits.
To evaluate the long-term effect of ABX464 on clinical response;
   Percentage of subjects reaching clinical response at yearly visits.
To evaluate the long-term effect of ABX464 on endoscopic improvement;
   Percentage of subjects reaching endoscopic improvement at yearly visits.
To evaluate the long-term effect of ABX464 on inflammatory markers (C-reactive protein (CRP), calprotectin);
   The change from Baseline in CRP and fecal calprotectin levels.
To evaluate the safety profile of ABX464;
   Number and rate of all AEs and causally-related AEs, all Serious Adverse Events (SAEs) and causally-related SAEs, all AEs of Special Interest (AESIs) and causally-related AESIs, classified by severity;
   Incidence of AEs leading to investigational medicinal product discontinuation;
   The number of clinically-significant laboratory abnormalities.
To evaluate the effect of ABX464 on miR-124 expression in total blood at every timepoint defined in the schedule of assessment.
   Change relative to baseline in miRNA-124 expression in total blood at every defined timepoint.

### Echocardiography Objective and Endpoints

To evaluate the effect of ABX464 on cardiac function as assessed through echocardiograms.
Absolute (%) change from baseline echocardiogram of left ventricular ejection fraction (LVEF) as measured by 2-dimensional echocardiography;
Number of subjects with a clinically relevant reduction (change from baseline echocardiogram) of LVEF, defined as >10% reduction (absolute percentage points) to a value<50%;
Absolute (%) change in left ventricular global longitudinal strain (LVGLS) from baseline echocardiogram;
Number of subjects with a relative percentage reduction in LVGLS by >15% from the previous value;
Number of subjects with a reduction of LVEF>10% (absolute percentage points) to a value≥50% with an accompanying fall in LVGLS>15%;
Number of subjects with reduction in LVEF by >10% (absolute percentage points) to a value≥50%;
Changes from baseline echocardiography of other echocardiographic parameters as described in the Statistical Analysis Plan (SAP), including 2-dimensional volumes, right ventricle size and systolic function and valve function.

### Study Specific Definitions

For the purpose of the study, the following definitions will be applied:
Clinical remission based on the Mayo Scoring system, is defined as stool frequency subscore=0 or 1 and rectal bleeding subscore=0 and endoscopy subscore=0 or 1 (modified to exclude friability).
Clinical Response is defined as a reduction from baseline in modified Mayo Score of at least 2 points and greater than or equal to 30 percent from baseline with an accompanying decrease in rectal bleeding sub-score of greater than or equal to 1 point or absolute rectal bleeding sub-score of less than or equal to 1 point.
Endoscopic remission defined as a Mayo endoscopic sub score of 0.
Endoscopic improvement is defined as a Mayo endoscopic sub score of ≤1 (excluding friability).

### Study Design and Methodology

This study is an open-label study aiming at evaluating the long-term safety and the efficacy profile of ABX464 given once a day (QD) at 25 mg in subjects who have been previously enrolled in the ABX464-102 or ABX464-104 studies (OLE and maintenance studies) and who are willing to continue their treatment.

All subjects will receive ABX464 given at 25 mg QD.

The enrollment in this long-term study will be based on the endoscopic improvement, the willingness of the subject to carry on his/her participation and also based on investigator's judgement.

Subjects will be treated with ABX464 for a maximum period of 54 months. Subjects will be followed up quarterly. After the treatment period, subjects will be followed for 4 additional weeks for safety purposes.

### Number of Subjects:

This study being the long-term follow-up study of 2 separate studies, there is no real calculation of the sample size. A maximum of 203 subjects will be included: subjects are those who have completed the open-label ABX464-102 or ABX464-104 studies and eligible to continue in this study.

### Inclusion Criteria:

A subject will be eligible to participate in this study if ALL the following criteria are met:
1. Subjects who previously completed the ABX464-102 or ABX464-104 clinical studies;
2. Subjects should be in endoscopic improvement with a rectal bleeding sub-score=0 point at the end of treatment period in the previous study (ABX464-102 or ABX464-104). Endoscopic improvement is defined as: a Mayo endoscopic sub score of ≤1;
3. Subjects able and willing to comply with study visits and procedures;
4. Subjects must understand, sign and date the written voluntary informed consent form at the visit prior to any protocol-specific procedures;
5. Women of childbearing potential and men receiving the study treatment and their partners must agree to continue a highly effective contraceptive method during the study and for at least 21 days after end of study or early termination. Women must be surgically sterile or if of childbearing potential must use a highly effective contraceptive method. Women of childbearing potential (WOCBP) will enter the study after confirmed menstrual period and a negative pregnancy test. The WOCB must be willing to perform once a month a urine pregnancy test. Highly effective methods of contraception include true abstinence, intrauterine device (IUD) or hormonal contraception aiming at inhibition of ovulation, intrauterine hormone releasing system, bilateral tubal ligation, vasectomized partner. True abstinence is defined when this is in line with the preferred and usual lifestyle of the subject This recommendation also applies to WOCBP with infrequent or irregular menstrual cycle. Female and male subjects must not be planning pregnancy during the trial and for at least 21 days post completion of their participation in the trial. In addition, male subjects should use condom during the trial and for at least 21 days post completion of their participation in the study. Male subjects must not donate sperm as long as contraception is required. For the purpose of this protocol, a woman is considered of childbearing potential (WOCBP), i.e. fertile, following menarche and until becoming post-menopausal unless permanently sterile. Permanent sterilization methods include hysterectomy, bilateral salpingectomy and bilateral oophorectomy. A postmenopausal state is defined as no menses for 12 months without an alternative medical cause. A high follicle stimulating hormone (FSH) level in the postmenopausal range may be used to confirm a post-menopausal state in women not using hormonal contraception or hormonal replacement therapy. Finally, a man is considered fertile after puberty unless permanently sterile by bilateral orchidectomy;
6. Subjects should be affiliated to a social security regimen (for French sites only).

### Exclusion Criteria:

Subjects who meet any of the following exclusion criteria will be excluded from the study:
1. Subjects who have developed any major illness/condition or evidence of an unstable clinical condition (except UC) that, in the investigator's judgment, will substantially increase the risk to the participant if he or she participates in the study;
2. Subjects with any other severe acute or chronic medical or psychiatric condition or laboratory or electrocardiogram (ECG) abnormality that may increase the risk associated with study participation or investigational product administration or may interfere with the interpretation of study results and, in the judgment of the investigator, would make the subject inappropriate for entry into this study;
3. Subjects who are participating or plan to participate in other investigational studies (other than induction study) during the study.

### Test Product, Dose, and Mode of Administration

ABX464 dose 25 mg is administrated once daily in fed condition (regular meal) in the morning with a glass of water.

Mandatory and/or Allowed Concomitant Medications
Oral 5-aminosalicylic acid at stable dose;
Immunosuppressants in the form of azathioprine, 6-mercaptopurine, or methotrexate at stable dose;
Antidiarrheals (e.g., loperamide, diphenoxylate with atropine) at stable dose;
Corticosteroids at stable dose of prednisone or prednisone equivalent≤15 mg/day; beclomethasone dipropionate (≤5 mg/day) or budesonide MMX (≤9 mg/day).

### Prohibited Concomitant Medications

Anti-tumor necrosis factor (TNF) therapies;
Vedolizumab;
JAK inhibitors;
Drugs that inhibit IL-12/23 (e.g., ustekinumab)
Topical corticosteroids and topical 5-aminosalicylic acid preparations;
Cyclosporine and tacrolimus;
Vaccination with live components during the study and up to 8 Weeks after the last dosing (of note, COVID vaccination is allowed with the exception of live-attenuated vaccine);
Drugs that strongly inhibit (ciprofloxacin, enoxacin, fluvoxamine) or induce (montelukast, phenytoin, rifampicin, ritonavir, teriflunomide) CYP1A2;
Drugs that inhibit UGTIA9 activity and inhibitors of OATP1B1/1B3 transporters;
Use of any investigational or non-registered product, except ABX464.

### Premature Trial Discontinuation

Subject's premature trial discontinuation must occur for the following reasons:
Investigator's decision;
An AE or an intercurrent condition that preclude continuation of treatment.
An increase≥3.0×ULN in liver transaminases (AST and/or ALT) or an increase≥2.0×ULN in alkaline phosphatase or in total bilirubin requires close observation with repeating liver enzymes and serum bilirubin tests twice weekly and clinical investigation to understand the etiology of this elevation. Frequency of retesting can decrease to once a month if abnormality stabilizes after the initial 2 weeks of follow-up and if the patient is asymptomatic. Discontinuation of the study treatment should occur if:
   ALT or AST>8×ULN;
   ALT or AST>5×ULN for more than 2 weeks;
   ALT or AST>3×ULN and total bilirubin>2×ULN or INR>1.5;
   ALT or AST>3×ULN with appearance of fatigue, nausea, vomiting, right upper quadrant pain or tenderness, fever, rash, and/or eosinophilia (>5%).
Worsening of the UC defined as a 2-point increase in the pMMS, with pMMS≥4 on 2 separate occasions 7 day-apart and confirmed by an endoscopy sub-score of 2 points or higher, subject who would experience a treatment failure during the study may be withdrawn at any time. S/he will have to be treated according to standard of care as soon as s/he is discontinued from study treatment because of study withdrawal;
Any cardiac AESI or any cardiac condition diagnosed during the course of the study and assessed by the Independent Cardiovascular Safety Committee as changing the risk/benefit balance for the subject;
Severe (grade 3 or higher) infection, a severe (grade 3 or higher) opportunistic infection, or sepsis;
Medically significant abnormal laboratory results, including new onset of anemia (defined as a hemoglobin decrease>2 g/dL from baseline or hemoglobin<8 g/dL) NOTE: transient abnormal values and/or abnormal values related to an existing condition that doesn't constitute a worsening will not be a criterion for discontinuation;
New onset of acute pancreatitis (as defined by Atlanta criteria);
Any malignancies, including dysplastic skin lesions;
Any relevant toxicity or negative change in the risk/benefit assessment leading to an unacceptable risk for the subject (i.e. occurrence of AEs which character, severity or frequency is new in comparison to the existing risk profile), or any data deriving from other clinical trials or toxicological studies which negatively influence the risk/benefit assessment;
Pregnancy;
Withdrawal of consent;
Administrative reasons from Sponsor.

### Subject Follow-Up

All subjects will be followed as per the site standard of care.

### Statistical Methods

### Safety:

Analysis of safety will be performed on the safety data set consisting in all subjects who received at least one dose of ABX464 in the study. The assessment of safety will be based on the frequency of AEs (with and without regard to causality) graded according to the “CTC-AE” (Version 5.0 or more recent version) and also, the review of individual values for clinical laboratory data and ECG focusing on the detection of abnormal values and potentially clinically significant abnormalities (PCSAs) determined upon investigator considerations.

Adverse Events will be tabulated (counts and percentages). All AEs will be listed, and the data will be tabulated by primary system organ class and preferred term, and will be further classified by severity, and relationship to treatment.

Clinical laboratory parameters and ECG will be summarized by using descriptive statistics (n, mean, SD, SEM, median, minimum and maximum). The number of subjects with at least one abnormal value will be tabulated (counts and percentages) for each parameter in summary shift tables.

### Efficacy:

Descriptive statistics will be presented for all secondary efficacy variables for each measurement timepoint. Continuous variables: mean, standard deviation, minimum and maximum, 95% confidence intervals, median and quartiles will be presented when considered relevant. Categorical variables: Counts, rates and 95% confidence intervals will be calculated.

In addition, an attempt will be made to compare the study groups in respect of changes from baseline in different measurements by mixed model analysis of covariance.

### End of Trial Definition

The study will end once all the recruited subjects have performed their safety follow-up. All the data will then be analyzed and computed in the clinical study report (CSR).

### Interim Analysis

The disease control rate after 48-week treatment with obefazimod 25 mg QD was analyzed for 74 patients, including 10 patients from the ABX464-102 LTE (4Y) and 64 patients from the ABX464-104 LTE (2Y). Among the 74 patients, 11 patients withdrew:
3 patients due to no eligibility (MES>1 at study baseline);
2 patients due to AE;
1 patient due to pregnancy;
5 patients due to consent withdrawal or investigator decision.

For the 63 patients who completed the 48-week treatment with obefazimod 25 mg QD, it was found:
Disease control Rate (Stable+improved MMS)=84.1%
Levels of Fecal Calprotectin consistent with the level of the disease control (93%<250 microgm/gm)

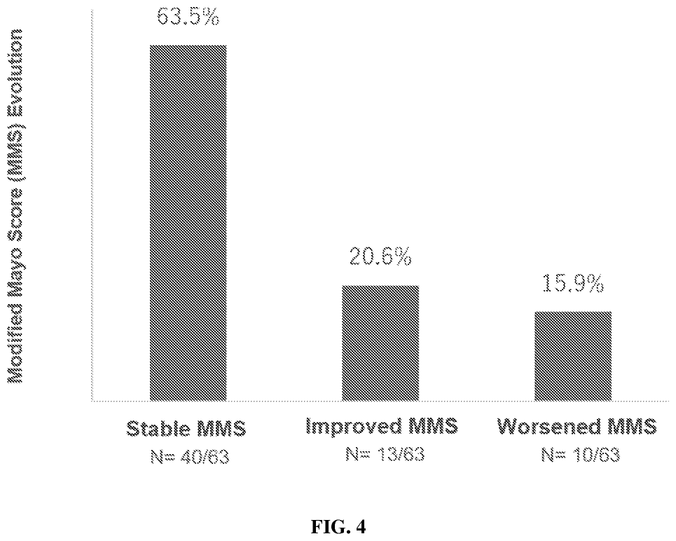

The MMS evaluation for the 63 patients after the 48-week treatment is depicted in FIG. 4. Obefazimod 25 mg QD continues to have durable efficacy in patients who achieved endoscopic improvement after 2-4 years of exposure to 50 mg Obefazimod. There was no new safety signals in UC patients treated with obefazimod for up to 5 years.

While we have described a number of embodiments of this invention, it is apparent that our basic examples may be altered to provide other embodiments that utilize the compounds and methods of this invention. Therefore, it will be appreciated that the scope of this invention is to be defined by the application and claims rather than by the specific embodiments that have been represented by way of example.

1.-36. (canceled)

37. A method for treating moderately to severely active ulcerative colitis comprising administering to a patient in need thereof a therapeutically effective amount of Obefazimod, or a pharmaceutically acceptable salt thereof, at an initial dose for an initial period, followed by a subsequent dose for a subsequent period,
wherein the initial dose is 25 mg or 50 mg Obefazimod administered once a day, and the initial period is 8 weeks,
wherein the subsequent dose is 25 mg or 50 mg Obefazimod administered once a day, and the subsequent period is at least 44 weeks, and
wherein during the initial period or the subsequent period, the patient exhibits clinical remission defined as having the following Mayo subscores: SFS=0 or 1 and RBS=0, and MES=0 or 1.

38. The method of claim 37, wherein during the initial period or the subsequent period, the patient exhibits at least one additional clinical endpoint selected from the following clinical endpoints:
i. endoscopic improvement defined as having the following Mayo subscores: MES=0 or 1,
ii. symptomatic remission defined as having the following Mayo subscores: SFS=0 or 1 (with a 1-point reduction from baseline) and RBS=0,
iii. clinical response defined as having a reduction from baseline in MMS≥2 points and a relative reduction from baseline in MMS≥30%, and a reduction from baseline in RBS≥1 point and/or RBS=0 or 1, and
histologic-endoscopic mucosal improvement defined as having endoscopic improvement associated with histologic improvement, wherein Histologic improvement is defined as having Geboes score≤3.1.

39. The method of claim 37, wherein the initial dose is 50 mg Obefazimod, administered once a day and the subsequent dose is 25 mg Obefazimod, administered once a day.

40. The method of claim 37, wherein clinical remission is observed at week 8 after the initial dose.

41. The method of claim 37, wherein the patient has an inadequate response, no response, a loss of response, or an intolerance to conventional therapies and/or advanced therapies for the treatment of moderately to severely active ulcerative colitis.

42. The method of claim 41, wherein the conventional therapy is a corticosteroid or an immunosuppressant.

43. The method of claim 42, wherein the immunosuppressant is selected from azathioprine, 6-mercaptopurine, and methotrexate.

44. The method of claim 41, wherein the advanced therapy is a biologic, a S1P receptor modulator, or a JAK inhibitor.

45. The method of claim 44, wherein the biologic is selected from TNF inhibitors, anti-integrins and anti-IL-23.

46. The method of claim 37, wherein clinical remission exhibited while administering the subsequent dose continues over a period greater than 12 months, greater than 18 months, greater than 24 months, greater than 30 months, greater than 36 months, greater than 42 months, greater than 48 months, greater than 54 months, or greater than 60 months.

47. The method of claim 38, wherein endoscopic improvement exhibited while administering the subsequent dose continues over a period greater than 12 months, greater than 18 months, greater than 24 months, greater than 30 months, greater than 36 months, greater than 42 months, greater than 48 months, greater than 54 months, or greater than 60 months.

48. The method of claim 38, wherein symptomatic remission exhibited while administering the subsequent dose continues over a period greater than 12 months, greater than 18 months, greater than 24 months, greater than 30 months, greater than 36 months, greater than 42 months, greater than 48 months, greater than 54 months, or greater than 60 months.

49. The method of claim 38, wherein clinical response exhibited while administering the subsequent dose continues over a period greater than 12 months, greater than 18 months, greater than 24 months, greater than 30 months, greater than 36 months, greater than 42 months, greater than 48 months, greater than 54 months, or greater than 60 months.

50. The method of claim 38, wherein histologic-endoscopic mucosal improvement exhibited while administering the subsequent dose continues over a period greater than 12 months, greater than 18 months, greater than 24 months, greater than 30 months, greater than 36 months, greater than 42 months, greater than 48 months, greater than 54 months, or greater than 60 months.

51. A method for treating moderately to severely active ulcerative colitis comprising administering to a patient in need thereof, a therapeutically effective amount of Obefazimod, or a pharmaceutically acceptable salt thereof, wherein Obefazimod is administered during an initial period of 8 weeks at an initial dose of 25 mg once a day or 50 mg once a day and during a subsequent period at a subsequent dose of 25 mg once a day or 50 mg once a day until the patient exhibits clinical remission, defined as having the following Mayo subscores: SFS=0 or 1 and RBS=0, and MES=0 or 1.

52. The method of claim 51, wherein during the initial period or the subsequent period, the patient exhibits at least one additional clinical endpoint selected from the following clinical endpoints:
i. endoscopic improvement defined as having the following Mayo subscores: MES=0 or 1,
ii. symptomatic remission defined as having the following Mayo subscores: SFS=0 or 1 (with a 1-point reduction from baseline) and RBS=0,
iii. clinical response defined as having a reduction from baseline in MMS≥2 points and a relative reduction from baseline in MMS≥30%, and a reduction from baseline in RBS≥1 point and/or RBS=0 or 1, and
histologic-endoscopic mucosal improvement defined as having endoscopic improvement associated with histologic improvement, wherein Histologic improvement is defined as having Geboes score≤3.1.

53. The method of claim 51, wherein the patient has an inadequate response, no response, a loss of response, or an intolerance to conventional therapies and/or advanced therapies for the treatment of moderately to severely active ulcerative colitis.

54. The method of claim 53, wherein the conventional therapy is a corticosteroid or an immunosuppressant.

55. The method of claim 54, wherein the immunosuppressant is selected from azathioprine, 6-mercaptopurine, and methotrexate.

56. The method of claim 53, wherein the advanced therapy is a biologic, a S1P receptor modulator, or a JAK inhibitor.

57. The method of claim 55, wherein the biologic is selected from TNF inhibitors, anti-integrins and anti-IL-2.

58. The method of claim 51, wherein clinical remission exhibited while administering the subsequent dose continues over a period greater than 12 months, greater than 18 months, greater than 24 months, greater than 30 months, greater than 36 months, greater than 42 months, greater than 48 months, greater than 54 months, or greater than 60 months.

59. The method of claim 52, wherein at least one additional clinical endpoint selected from endoscopic improvement, symptomatic remission, clinical response, and histologic-endoscopic mucosal improvement exhibited while administering the subsequent dose continues over a period greater than 12 months, greater than 18 months, greater than 24 months, greater than 30 months, greater than 36 months, greater than 42 months, greater than 48 months, greater than 54 months, or greater than 60 months.

60. A method for treating moderately to severely active ulcerative colitis in a patient comprising administering to a patient in need thereof a therapeutically effective amount of Obefazimod, or a pharmaceutically acceptable salt thereof, at an initial dose for an initial period, followed by a subsequent dose for a subsequent period,
wherein the initial dose is 25 mg or 50 mg Obefazimod administered once a day, and the initial period is 8 weeks,
wherein the subsequent dose is 25 mg or 50 mg Obefazimod administered once a day, and the subsequent period is at least 44 weeks, and
wherein at week 8 the patient exhibits at least one clinical endpoint selected from the following clinical endpoints:
i. clinical remission defined as having the following Mayo subscores: SFS=0 or 1 and RBS=0, and MES=0 or 1,
ii. endoscopic improvement defined as having the following Mayo subscores: MES=0 or 1,
iii. symptomatic remission defined as having the following Mayo subscores: SFS=0 or 1 (with a 1-point reduction from baseline) and RBS=0,
iv. clinical response defined as having a reduction from baseline in MMS≥2 points and a relative reduction from baseline in MMS≥30%, and a reduction from baseline in RBS≥1 point and/or RBS=0 or 1, and
histologic-endoscopic mucosal improvement defined as having endoscopic improvement associated with histologic improvement, wherein Histologic improvement is defined as having Geboes score≤3.1.

61. A method for treating moderately to severely active ulcerative colitis comprising administering to a patient in need thereof, a therapeutically effective amount of Obefazimod, or a pharmaceutically acceptable salt thereof, at an initial dose for an initial period, followed by a subsequent dose for a subsequent period,
wherein the initial dose is 25 mg or 50 mg Obefazimod administered once a day, and the initial period is 8 weeks,
wherein the subsequent dose is 25 mg or 50 mg Obefazimod administered once a day, and the subsequent period is at least 44 weeks, and
wherein during the initial period or the subsequent period, the patient exhibits a fecal calprotectin level of less than 250 mcg/g.

62. The method of claim 61, wherein during the initial period or the subsequent period, the patient exhibits a fecal calprotectin level of less than 150 mcg/g.

63. The method of claim 61, wherein a fecal calprotectin level of less than 250 mcg/g exhibited while administering the subsequent dose continues over a period greater than 12 months, greater than 18 months, greater than 24 months, greater than 30 months, greater than 36 months, greater than 42 months, greater than 48 months, greater than 54 months, or greater than 60 months.

64. The method of claim 60, wherein the patient has an inadequate response, no response, a loss of response, or an intolerance to conventional therapies and/or advanced therapies for the treatment of moderately to severely active ulcerative colitis.

65. The method of claim 64, wherein the conventional therapy is a corticosteroid or an immunosuppressant.

66. The method of claim 65, wherein the immunosuppressant is selected from azathioprine, 6-mercaptopurine, and methotrexate.

67. The method of claim 64, wherein the advanced therapy is a biologic, a S1P receptor modulator, or a JAK inhibitor.

68. The method of claim 67, wherein the biologic is selected from TNF inhibitors, anti-integrins and anti-IL-23.
